(19) United States
(12) Patent Application Publication
(10) Pub. No.: US 2003/0124526 A1
(43) Pub. Date: Jul. 3, 2003

(54) Polymer synthesizer

(75) Inventors: Raymond F. Cracauer, Middleton, WI (US); Zbigniev Skrzypczynski, Verona, WI (US); Ned D. Reimer, Madison, WI (US)

Correspondence Address: MEDLEN & CARROLL, LLP, 101 HOWARD STREET, SUITE 350, SAN FRANCISCO, CA, 94105, US

(73) Assignee: Third Wave Technologies, Inc.

(21) Appl. No.: 09/930,688

(22) Filed: Aug. 15, 2001

(51) Int. Cl. C12Q 1/68; C07H 21/02; C07H 21/04; C12M 1/34

(52) U.S. Cl. 435/6; 536/25.3; 435/287.2

(57) ABSTRACT

The present invention relates to polymer synthesizers and methods of using polymer synthesizers. For example, the present invention provides highly efficient, reliable, and safe synthesizers that find use, for example, in high throughput and automated nucleic acid synthesis. The present invention also relates to synthesizer arrays for efficient, safe, and automated processes for the production of large quantities of polymers.

### FIELD OF THE INVENTION

The present invention relates to polymer synthesizers and methods of using polymer synthesizers. For example, the present invention provides highly efficient, reliable, and safe synthesizers that find use, for example, in high throughput and automated nucleic acid synthesis (e.g., automated synthesis of modified and unmodified nucleic acids and their conjugates). The present invention also relates to synthesizer arrays for efficient, safe, and automated processes for the production of large quantities of polymers.

### BACKGROUND

With the completion of the Human Genome Project and the increasing volume of genetic sequence information available, genomics research and subsequent drug design efforts have been increasing as well. Many diagnostic assays and therapeutic methods utilize oligonucleotides (e.g., DNA or RNA oligonucleotides, modified oligonucleotides and their conjugates). The information obtained from genomic analysis provides valuable insight into the causes and mechanisms of a large variety of diseases and conditions, while oligonucleotides can be used to alter gene expression in cells and tissues to prevent or attenuate diseases or alter physiology. As more nucleic acid sequences continue to be identified, the need for larger quantities of oligonucleotides used in assays and therapeutic methods increases.

To meet the increasing demand for nucleic acid synthesis, there has been an increase in the variety of designs, and the volume of production of nucleic acid synthesizers. Unfortunately, the currently available synthesizers are not designed to adequately meet the needs of the industry. In particular, available synthesizers are limited in their ability to efficiently synthesize large numbers of oligonucleotides. While synthesizers have been developed to simultaneously synthesize more than one oligonucleotide at a time, such machines are not efficient at the production of different types of nucleic acids simultaneously (e.g., different lengths of nucleic acids) and are unacceptably prone to performance failures and environmental contamination. Furthermore, available synthesizes are not suitably configured for use in large-scale nucleic acid production facilities or for automated nucleic acid synthesis. Thus, the art is in need of nucleic acid synthesizers that are safe, efficient, flexible, and are amenable to large-scale production and automation.

### SUMMARY OF THE INVENTION

The present invention relates to polymer synthesizers and methods of using polymer synthesizers. For example, the present invention provides highly efficient, reliable, and safe synthesizers that find use, for example, in high throughput and automated nucleic acid synthesis. The present invention also relates to synthesizer arrays for efficient, safe, and automated processes for the production of large quantities of oligonucleotides.

For example, the present invention provides a system comprising a closed system solid phase synthesizer configured for parallel synthesis (e.g., simultaneous side-by-side synthesis) of three or more polymers (e.g., 3, 4, 5, 6, 7, . . . , 10, . . . , 48, . . . , 96, . . . ). The present invention is not limited by the nature of the polymer. Polymers include, but are not limited to, nucleic acids and polypeptides. In some preferred embodiments, the nucleic acid polymers comprise DNA. In some particularly preferred embodiments, the DNA comprises an oligonucleotide.

The synthesizers of the present invention allow parallel synthesis of multiple polymers. Each of the synthesized polymers may be identical to one another (e.g. in composition, sequence, length, etc.) or may be different than one another (e.g., in composition, sequence, length, etc.). Thus, the synthesizers of the present invention may be configured to simultaneously produce three or more distinct polymers (e.g., oligonucleotides).

Because the synthesizers of the present invention allow parallel processing of polymers, large numbers of polymers may be produced in a single synthesizer in a short period of time. For example, the synthesizer may be configured to produce 100 or more polymers per day. In some embodiments, the synthesizer may be configured to produce 1000-2000 or more polymers per day. For example, synthesizers may be configured to produce 2000 or more oligonucleotide per day (e.g., oligonucleotides containing 20-40 or more bases). In some preferred embodiments, the produced polymers (e.g., 2000 or more produced polymers) are produced at a 1 μM synthesis scale. In some embodiments, the produced polymers are produced on a micro-scale, e.g., less than 5 nmole synthesis scale. In some preferred embodiments, micro-scale synthesis is performed on a 0.1 to 1 nmole synthesis scale.

The present invention also provides a solid phase synthesizer comprising: a reaction support comprising three or more (e.g., 3, 4, 5, 6, 7, . . . , 10, . . . , 48, . . . , 96, . . . ) reaction chambers (e.g., chambers that are isolated from one another, such that fluid does not pass from one chamber to another during synthesis); and a plurality of reagent dispensers configured to simultaneously form closed fluidic connections with each of the reaction chambers, wherein the reagent dispensers are each configured to deliver all reagents necessary for a polymer synthesis reaction. In some embodiments, the reaction chambers comprise synthesis columns. For example, the reaction support provides a fixed surface to support three or more synthesis columns. In some embodiments, the synthesis columns comprise nucleic acid synthesis columns (e.g., columns designed for use with EXPEDITE nucleic acid synthesizers [Applied Biosystems, Foster City, Calif.], 3900 High-Throughput Columns for use with the 3900 DNA Synthesizer [Applied Biosystems], DNA synthesis columns from Biosearch Technologies, Novato, Calif.). In preferred embodiments, the reaction support is configured to contain and form a tight seal around multiple, different synthesis columns (e.g., of different sizes or from different manufacturers), so as to allow any number of commercially available columns to be used with the synthesizer.

In some embodiments, the reagent dispensers are fluidicly connected to a plurality of reagent tanks (e.g., through tubing). In preferred embodiments, reagent dispensers are constructed from any substantially inert materials including, but not limited to, stainless steel, glass, Teflon, and titanium. Tanks include, but are not limited to, acetonitrile tanks, phosphoramidite tanks, argon gas tanks, oxidizer tanks, tetrazole tanks, and capping solution tanks. In some embodiments, the tanks are contained within the synthesizer. In other embodiments, the tanks are contained on an outer surface of the synthesizer. In some preferred embodiments, tanks are provided separately from the synthesizer (e.g., in a different room, such as an explosion-proof room). For example, in some embodiments, the present invention provides large volume synthesis facilities containing multiple synthesizers, wherein two or more of the synthesizer are serviced by the same reagent tanks. In some such embodiments, “large volume containers” are used as reagent tanks. Individual large volume reagent tanks contain from about 200 liters to about 2500 liters of acetonitrile, from about 200 liters to about 2500 liters of deblocking solution; from about 2 liters to about 200 liters of amidite; from about 20 liters to about 200 liters of activator (e.g., tetrazol); from about 20 liters to about 200 liters of capping reagents; or from about 20 liters to about 200 liters of oxidizer. Alternatively, a plurality of tanks containing a combined capacity as indicated above may be used. In some embodiments, the large volume reagent tanks are connected to a plurality of synthesizers through a large volume reagent delivery system, which allows large volumes of reagents to be delivered simultaneously to each of the synthesizers

Various useful reagents and coupling chemistries are described in U.S. Pat. No. 5,472,672 to Bennan, and U.S. Pat. No. 5,368,823 to McGraw et al. (both of which are herein incorporated by reference in their entireties). In addition to phosphoramidite chemistries, phosphate and phosphite triester methods, and H-phosphonate methods of oligonucleotide synthesis are contemplated.

In some embodiments, the reaction support comprises a fixed reaction support (e.g., a reaction support that does not move during operation). In some embodiments, the reaction support comprises a plurality of waste channels. In preferred embodiments, the waste channels in closed fluidic contact with each of the reaction chambers (See e.g., FIG. 1).

In some embodiments, the synthesizer further comprises providing energy, such as heat to the reaction chambers. Heating of the reaction chamber finds use, for example, in decreasing the coupling time during a nucleic acid synthesis. It can also broaden the range of the chemical protocols that can be used in high throughput synthesis, e.g. by improving the efficiency of less efficient chemistries, such as the phosphate triester method of oligonucleotide synthesis. In other embodiments, the synthesizer further comprises a mixing component, such as an agitator, configured to agitate the reaction chambers (e.g., to mix reaction components, and to facilitate mass exchange between the reaction medium and the solid support).

The present invention further provides a solid phase synthesizer comprising: a fixed reaction support comprising three or more reaction chambers; and a plurality of reagent dispensers configured to simultaneously form closed fluidic connections with each of said reaction chambers.

The present invention also provides integrated systems that link nucleic acid synthesizers to other nucleic acid production components. For example, the present invention provides a system comprising a closed system nucleic acid synthesizer and a cleavage and deprotect component. In some embodiments, the synthesizer is configured for parallel synthesis of nucleic acid molecules at three or more reaction sites. In some preferred embodiments, the system further comprises a reaction support comprising three or more reaction chambers, wherein the reaction support is configured for operation with both the nucleic acid synthesizer and the cleavage and deprotect component. In some embodiments, the system further comprises sample tracking software configured to associate sample identification tags (e.g., electronic identification numbers, barcodes) with samples that are processed by the nucleic acid synthesizer and the cleavage and deprotect component. In some preferred embodiments, the sample tracking software is further configured to receive synthesis request information from a user, prior to sample processing by the nucleic acid synthesizer. In some embodiments, the system further comprises a robotic component configured to transfer the reaction support from the nucleic acid synthesizer to the cleavage and deprotect component. In other preferred embodiments, the robotic component is further configured to transfer the reaction support from the cleavage and deprotect component to a purification component and/or to additional production components described herein.

The present invention also provides control systems for operating one or more components of the systems of the present invention. For example, the present invention provides a system comprising a processor, wherein the processor is configured to operate a close system nucleic acid synthesizer for parallel synthesis of three or more nucleic acid molecules. The present invention further provides a system comprising a processor, wherein said processor is configured to operate a nucleic synthesizer and a cleavage and deprotect component. In some embodiments, the system further comprises a computer memory, wherein the computer memory comprises nucleic acid sample order information (e.g., information obtained from a user specifying the identity of a polymer to be synthesized and/or specifying one or more characteristics of the polymer such as sequence information). In some embodiments, the computer memory further comprises allele frequency information and/or disease association information.

### DESCRIPTION OF THE DRAWINGS

FIG. 1 shows a schematic diagram of a polymer synthesizer of the present invention.

FIG. 2A shows a side view of a reagent dispenser (2).

FIG. 2B shows a cross-sectional view of a reagent dispenser (2)

FIG. 3A shows an embodiment of a reagent dispenser having a first (13) and a second (14) ring seal.

FIG. 4A shows a synthesizer having a reagent dispensing station as an integral part of the base (16)

FIG. 4A shows a synthesizer having panels providing an enclosed reagent dispensing station.

FIG. 5 shows a solvent delivery component in one embodiment of the present invention.

FIG. 6 shows a waste storage and purge component in one embodiment of the present invention.

### DEFINITIONS

To facilitate an understanding of the present invention, a number of terms and phrases are defined below: As used herein, the term “synthesis” refers to the assembly of polymers from smaller units, such as monomers.

As used herein, the term “fluidic connection” refers to a continuous fluid path between components.

As used herein, the term “parallel” refers to systems or actions functioning in an essentially simultaneous, side-by-side, manner (e.g., parallel synthesis or parallel synthesis system).

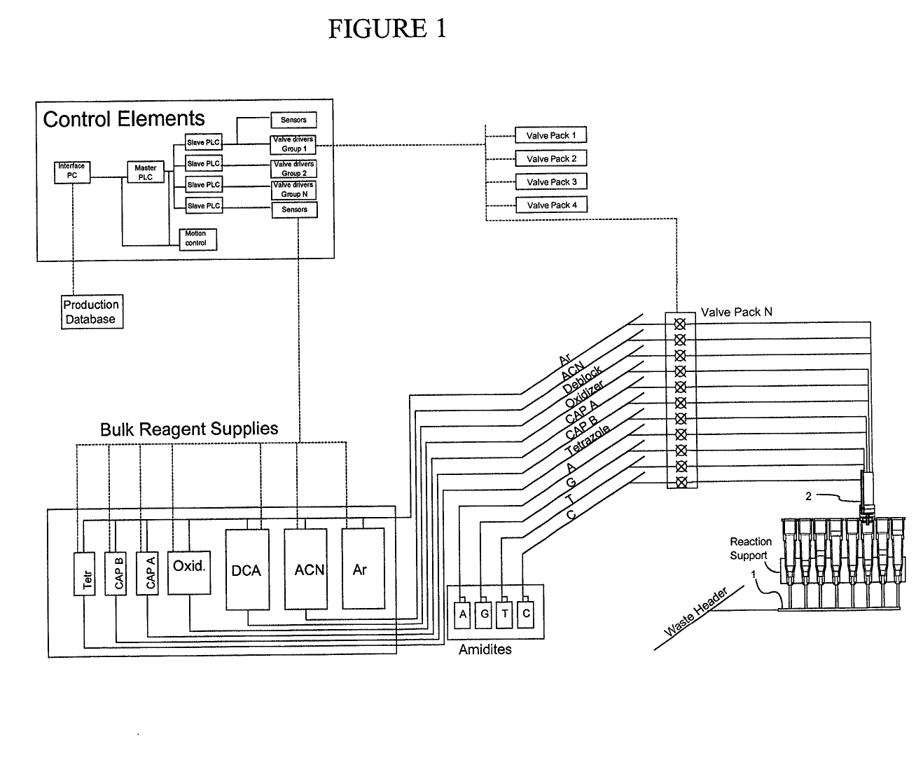

As used herein, the term “reaction support” refers to a structure supporting, comprising, or containing one or more reaction chambers (see, e.g., FIG. 1).

As used herein, the terms “centralized control system” or “centralized control network” refer to information and equipment management systems (e.g., a computer processor and computer memory) operable linked to or integrated into a module or modules of equipment (e.g., DNA synthesizer or a computer operably linked to a DNA synthesizer).

As used herein the terms “processor” and “central processing unit” or “CPU” are used interchangeably and refer to a device that is able to read a program from a computer memory (e.g., ROM or other computer memory) and perform a set of steps according to the program.

As used herein, the terms “computer memory” and “computer memory device” refer to any storage media readable by a computer processor. Examples of computer memory include, but are not limited to, RAM, ROM, computer chips, digital video disc (DVDs), compact discs (CDs), hard disk drives (HDD), flash (solid state) recording media and magnetic tape.

As used herein, the term “cartridge” refers to a device for holding one or more synthesis columns. For example, cartridges can contain a plurality of openings (e.g., receiving holes) into which synthesis columns may be placed. “Rotary cartridges” refer to cartridges that, in operation, can rotate with respect to an axis, such that a synthesis column is moved from one location in a plane (a reagent dispensing location) to another location in the plane (a non-reagent dispensing location) following rotation of the cartridge.

As used herein, the term “nucleic acid synthesis column” refers to a container or chamber in which nucleic acid synthesis reactions are carried out. For example, synthesis columns include plastic cylindrical columns and pipette tip formats, containing openings at the top and bottom ends. The containers may contain or provide one or more matrices, solid supports, and/or synthesis reagents necessary to carry out chemical synthesis of nucleic acids. For example, in some embodiments of the present invention, synthesis columns contain a solid support matrix on which a growing nucleic acid molecule may be synthesized. Nucleic acid synthesis columns may be provided individually; alternatively, several synthesis columns may be provided together as a unit, e.g., in a strip or array, or as device such as a plate having a plurality of suitable chambers. Columns may be constructed of any material or combination of materials that do not adversely affect (e.g., chemically) the synthesis reaction or the use of the synthesized product. For example, columns or chambers may comprise polymers such as polypropylene, fluoropolymers such as TEFLON, metals and other materials that are substantially inert to synthesis reaction conditions, such as stainless steel, gold, silicon and glass. In some embodiments, chambers comprise a coating of such a suitable material over a structure comprising a different material.

As used herein, the term “seal” refers to any means for preventing the flow of gas or liquid through an opening. For example, a seal may be formed between two contacted materials using grease, o-rings, gaskets, and the like. In some embodiments, one or both of the contacted materials comprises an integral seal, such as, e.g., a ridge, a lip or another feature configured to provide a seal between said contacted materials. An “airtight seal” or “pressure tight seal” is a seal that prevents detectable amounts of air from passing through an opening. A “substantially airtight” seal is a seal that prevents all but negligible amounts of air from passing through an opening. Negligible amounts of air are amounts that are tolerated by the particular system, such that desired system function is not compromised. For example, a seal in a nucleic acid synthesizer is considered substantially airtight if it prevents gas leaks in a reaction chamber, such that the gas pressure in the reaction chamber is sufficient to purge liquid in synthesis columns contained in the reaction chamber following a synthesis reaction. If gas pressure is depleted by a leak such that the purge of the synthesis columns is affected (for example, if the synthesis columns are not purged, resulting, e.g., in overflow during subsequent synthesis rounds), then the seal is not a substantially airtight seal. A substantially airtight seal can be detected empirically by carrying out synthesis and checking for failures (e.g., column overflows) during one or a series of reactions.

As used herein, the term “sealed contact point” refers to sealed seams between two or more objects. Seals on sealed contact points can be of any type that prevent the flow of gas or liquid through an opening. For example, seals can sit on the surface of a seam (e.g., a face seal) or can be placed within a seam, such that a circumferential contact is created within the seam.

As used herein, the term “alignment detector” refers to any means for detecting the position of an object with respect to another object or with respect to the detector. For example, alignment detectors may detect the alignment of a dispensing end of a dispensing device (e.g., reagent channel, a waste channel, etc.) to a receiving device (e.g., a synthesis column, a waste valve, etc.). Alignment detectors may also detect the tilt angle of an object (e.g., the angle of a plane of an object with respect to a reference plane). For example, the tilt angle of a plate mounted on a shaft may be detected to ensure a proper perpendicular relationship between the plate and the shaft. Alignment detectors include, but are not limited to, motion sensors, infra-red or LED-based detectors, and the like.

As used herein, the term “motor connector” refers to any type of connection between a motor and another object. For example a motor designed to rotate another object may be connected to the object through a metal shaft, such that the rotation of the shaft, rotates the object. The metal shaft would be considered a motor connector.

As used herein, the term “packing material” refers to material placed in a passageway (e.g., a synthesis column) in a manner such that it provides resistance against a pressure differential between the two ends of the passageway (i.e. hinders the discharge of the pressure differential). Packing material may comprise a single material or multiple materials. For example, in some embodiments of the present invention, packing material comprising a nucleic acid synthesis matrix (e.g., a solid support for nucleic acid synthesis such as controlled pore glass, polystyrene, etc.) and/or one or more frits are used in synthesis columns to maintain a pressure differential between the two ends of the synthesis column. Packing material may be distributed into the reaction chambers in a variety of forms. For example, synthesis support matrix may be provided as a granular powder. In some embodiments, support matrix may be provided in a “pill” form, wherein an appropriate amount of a support material is held together with a binder to form a pill, and wherein one or more pills are provided to a reaction chamber, as appropriate for the scale of the intended reaction, and further wherein the binder is removed or inactivated (e.g., during a wash step) to allow the powdered matrix to function in the same manner as an unbound powder. The use of a pill embodiment provides the advantages of facilitating the process of pre-measuring synthesis support materials, allowing easy storage of support matrices in a pre-measured form, and simplifying provision of measured amounts of synthesis support matrix to a reaction chamber.

As used herein, the term “idle,” in reference to a synthesis column, refers to columns that do not take part in a particular synthesis reaction step of a nucleic acid synthesizer. Idle synthesis columns include, but are not limited to, columns in which no synthesis occurs at all, as well as columns in which synthesis has been completed (e.g., for short oligonucleotide) while other columns are actively undergoing additional synthesis steps (e.g., for longer oligonucleotides).

As used herein, the term “active,” in reference to a synthesis column, refers to columns that take part (or are taking part) in a particular synthesis reaction step of a nucleic acid synthesizer. Active synthesis columns include, but are not limited to, columns in which liquid reagents are being dispensed into, or columns that contain liquid reagents (e.g. waiting to be purged), or columns that are in the process of being purged.

As used herein, the term “alignment markers” refers to reference points on an object that allow the object to be aligned to one or more other objects. Alignment markers include pictorial markings (e.g., arrows, dots, etc.) and reflective markings, as well as pins, raised surfaces, holes, magnets, and the like.

As used herein, the term “O-ring” refers to a component having a circular or oval opening to accommodate and provide a seal around another component having a circular or oval external cross-section. An O-ring will generally be composed of material suitable for providing a seal, e.g., a resilient air-or moisture-proof material. In some embodiments, an O-ring may be a circular opening in a larger gasket. A single gasket may contain multiple openings and thus provide multiple O-rings. In other embodiments, an O-ring may be ring-shaped, i.e., it may have circular interior and exterior surfaces that are essentially concentric.

As used herein, the term “viewing window” refers to any transparent component configured to allow visual inspection of an item or material through the window. An enclosure may include a transparent portion that provides a viewing window for item within the disclosure. Likewise, an enclosure may be made entirely of a transparent material. In such embodiments, the entire enclosure can be considered a viewing window. A “viewing window” in an enclosure that is “configured to allow visual inspection” of items in the enclosure “without opening the enclosure” refers to a viewing window in an enclosure of sufficient size, location, and transparency to allow the item to be viewed, unhindered, by the human eye. For example, where the item is one or more reagent bottles, the window is configured to allow viewing of the reagents bottles by the human eye to determine if the bottles or full or empty. A window that does not provide adequate visual inspection of each of the reagent bottles is not configured to allow visual inspection of reagents in the enclosure without opening the enclosure.

As used herein, the term “enclosure” refers to a container that separates materials contained in the enclosure from the ambient environment. For example, an enclosure may be used with a reagent station to contain reagents within an interior chamber of the enclosure, and therefore separate the reagents from the ambient environment. In some embodiments, the enclosure provides an airtight or substantially airtight seal between the interior and exterior of the enclosure. The enclosure may contain one or more valves (e.g., ventilation ports), doors, or other means for allowing gasses or other materials (e.g., reagent bottles) to enter or leave the interior environment of the enclosure.

### DETAILED DESCRIPTION OF THE INVENTION

The present invention provides polymer synthesizers that permit parallel synthesis of large numbers of polymers. The following description provides illustrative examples of synthesizers of the present invention and is not intended to limit the scope thereof. While the description provided below focuses on the example of nucleic acid synthesis, it will be appreciated that the systems and methods are generally applicable to the synthesis of other polymers.

In preferred embodiments, the present invention provides closed-system solid phase synthesizers that are suitable for use in large-scale polymer production facilities. Each synthesizer is itself capable of producing large volumes of polymers. However, the present invention provides systems for integrating multiple synthesizers into a production facility, to further increase production capabilities. The description is provided in the following sections: I) Synthesizers and II) Production Facilities.

I. Synthesizers

Currently available nucleic acid synthesizers have limited synthesis capacity. For example, the 3900 DNA Synthesizer (Applied Biosystem, Foster City, Calif.) is one of the most capable synthesizers and produces fewer than 100 40-mer oligonucleotides in a typical day production run. Additional synthesizers are described in U.S. Pat. Nos. 5,744,102, 4,598,049, 5,202,418, 5,338,831, 5,342,585, 6,045,755, and 6,121,054, and PCT publication WO 01/41918, herein incorporated by reference in their entireties.

The synthesizers of the present invention dramatically increase capacity, with some embodiments allowing over 2000 40-mer oligonucleotides to be produced per day (e.g., during a 16 hour production day) at a 1 μM scale. These capacities are achieved through the use of multi-chamber reaction supports that allow parallel synthesis of polymers within each chamber. For example, three or more chambers (e.g., comprising synthesis columns), preferably 96 or more chambers are provided on a reaction support, permitting a plurality of different oligonucleotides to be simultaneously produced. Each reaction chamber is associated with its own reagent dispenser such that reagents are delivered to each chamber substantially simultaneously rather than delivery reagents in sequence. In preferred embodiments, the synthesizer is a closed system during operation (i.e., reagent delivery to the chambers and waste removal from the chambers occurs in a continuous pathway that is isolated from the ambient environment). An example of a closed system is illustrated in FIG. 1. In some preferred embodiments, the synthesizers have a minimum number of moving parts. In particular, the reaction support is immobile.

In some embodiments, the synthesizer provides additional polymer production capabilities. For example, in some embodiments, the synthesizer is configured to conduct cleavage and deprotection of synthesized oligonucleotide. In preferred embodiments, the same reaction support is used for both synthesis and cleavage and deprotection. In other preferred embodiments, the same reagent dispensers are used for both synthesis and cleavage and deprotection. In still other preferred embodiments, the reaction support does not move during both the synthesis and cleavage and deprotection processes (i.e., synthesis and cleavage and deprotection occur at the same location). In some embodiments, the synthesizer also provides an integrated purification component (e.g., using the same reaction support and/or reagent dispensers with or without movement of the reaction support). Any other production components described herein may also be integrated with the synthesizer.

Preferred features of the synthesizers of the present invention include: single day synthesis capacities of 2000 oligonucleotides, based on an average 40-mer at 1 μM scale with 16 hours staffing; production scale capabilities of 40, 100, 1000, and 4000 nM, with larger scales supported by control elements; compatibility with commercially available nucleic acid synthesis columns (e.g., columns designed for use with EXPEDITE nucleic acid synthesizers [Applied Biosystems, Foster City, Calif.], 3900 High-Throughput Columns for use with the 3900 DNA Synthesizer [Applied Biosystems], DNA synthesis columns from Biosearch Technologies, Novato, Calif.); mechanical and/or data interface capability with other production components (see Section II, below); individual oligonucleotide tracking (e.g., during synthesis and throughout an entire production process); compatibility with standard nucleic acid synthesis chemistry with provisions for optimization of reaction conditions; detectors for monitoring trityl or other components or reagents; compatibility with standard multi-chamber formats (e.g., 96-well plate, 384-well plate formats); interface with databases to input and track information including, but not limited to oligonucleotide sequence, completion, data, time, and channel; and integration with a control system to allow multiple synthesizers to have a common control center.

Reagent delivery to the synthesizer is achieved using a novel fluidics system. In preferred embodiments, all fluid transfers are desired to be closed system; that is, a closed fluid circuit exists from source to waste at any time reagents are being transferred. In general, the supply circuit remains coupled to the synthesis columns that are supported by the reaction support for all operations except, in some embodiments, during nucleic acid coupling reactions. Given the reaction time required for the coupling reactions (approximately 30 seconds), in some embodiments, the circuit to a particular column or columns is disconnected to allow fluid transfer mechanisms to be used on other columns. While the fluid transfer is re-routed, the columns undergoing the coupling reaction need not be exposed to the ambient environment (i.e., a sealed delivery path may be maintained).

In preferred embodiments, the target fluid transfer system is a pressurized supply with dispense control valves. Reagents flow to the reaction chambers upon opening of the control valves, driven by a pressure differential.

In some preferred embodiments, the reaction support contains waste channels configured to receive waste from the reaction chambers. In some embodiments, each channel is configured with its own waste channel (See e.g., FIG. 1). The waste channels preferably feed into a single waste disposal line. In some embodiments, the waste system is gravity driven. In other embodiments, a valve-controlled vacuum is used to eliminate waste. In some preferred embodiments, waste lines are fitted with a trityl monitoring device. In preferred embodiments, the waste line is fitted with aqualitative trityl monitoring device. For example, colorimetric analysis of effluent using a CCD camera or a similar device provides a yes/no answer on a particular detritylation level. Qualitative detection of detritylation can generally be performed with less expensive equipment than is generally required by more precise quantitation, and yet generally provides sufficient monitoring for detritylation failure. Valves used to control reagent delivery and/or waste removal may be under automated control.

In preferred embodiments, a plurality of reagent dispensers are provided, wherein a reagent dispenser is provided for each reaction chamber. In such embodiments, the reagent dispensers provide each of the reagents necessary to support a synthesis reaction within the reaction chamber. For nucleic acid synthesis, this includes, for example, delivery of acetonitrile, phosphoramidite corresponding to each of the bases, argon gas, oxidizer, activator (e.g., tetrazole), deblocking solution and capping solution. Thus, in some embodiments, the reagent dispenser comprises a plurality of reagent delivery lines, each line providing a direct fluidic connection between the reagent dispenser and individual supply tanks for the different reagents (See e.g., FIG. 1).

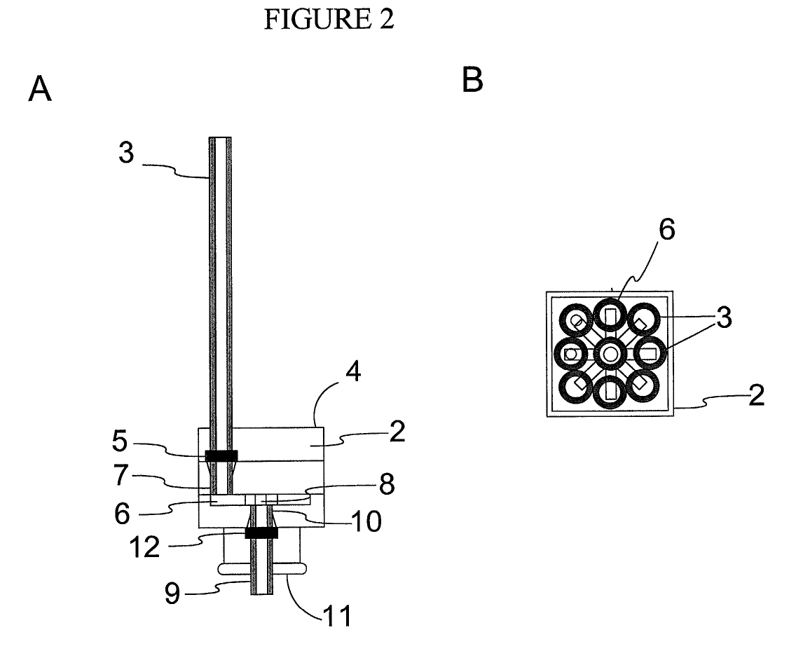

An example of such a reagent dispenser (2) is shown in FIG. 2 from both a side view (FIG. 2A) and a cross-sectional bottom view (FIG. 2B). The side view shows a single reagent delivery line (3) penetrating a top surface (4) of the reagent dispenser (2). In this embodiment, a retention ring (5) is used to support the reagent delivery line (3). The reagent delivery line (3) ends at a reagent reservoir (6) that is configured to receive reagents from each of the delivery lines. A seal (7) forms a contact between the delivery line (3) and the reagent reservoir (6). The center of the reagent reservoir (6) comprises a delivery aperture (8). The delivery aperture (8) is in fluidic contact with a delivery channel (9), with a seal (10) forming a contact between the delivery channel (9) and the delivery aperture (8). The delivery channel (9) passes through a bottom surface (11) of the reagent dispenser (2) and may positioned by a retention ring (12).

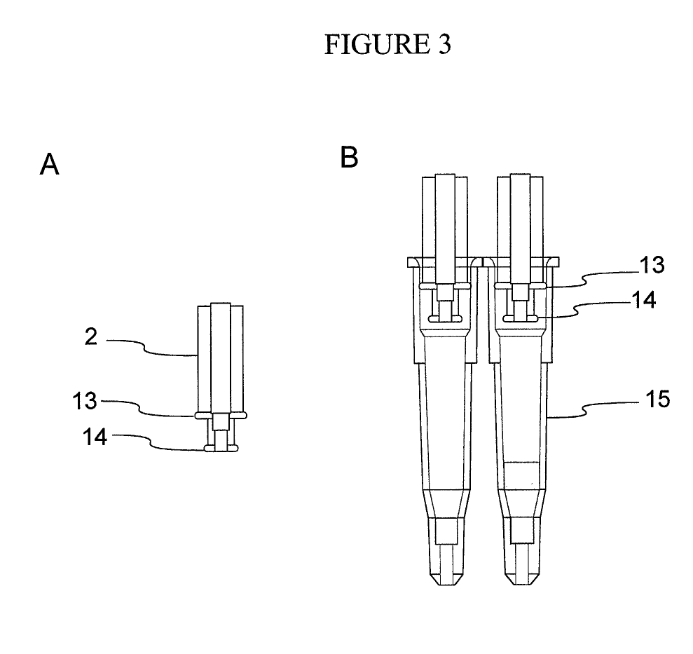

The cross-sectional bottom view shown in FIG. 2B shows the presence of nine delivery lines (3) contained within the reagent dispenser (2). Each delivery line empties into the reagent reservoir (6), represented by the eight pronged star. FIG. 3A shows one preferred embodiment of the reagent dispenser (2), wherein the outer surface of the delivery channel (9) contains first (13) and second (14) ring seals configured to form an airtight or substantially airtight seal with one or more points on the interior surface of a synthesis column (15) or other reaction chamber (e.g., with reaction chambers present in a synthesizer or a cleavage and deprotection component; see, for example FIG. 3B).

In preferred embodiments, common reagent tanks supply reagents to all of the reaction chambers. The reagents tanks may be contained within the synthesizer or may be external to the synthesizer. Where the tanks are provided with the synthesizer, they are preferably contained in a vented chamber to reduce the build-up of gaseous or liquid waste in and around the synthesizer. In some preferred embodiments, common reagent tanks supply reagents to a plurality of synthesizers. Examples of such delivery systems are provided in Section II, below. In yet other embodiments, some of the reagents are supplied externally and some of the reagents are supplied at or in the synthesizer (e.g., amidites). In some embodiments, one or more of the reagents are processed, e.g., under vacuum, to remove dissolved gasses.

In some preferred embodiments, the synthesizer comprises a means of delivering energy to the reaction chambers to, for example, increase nucleic acid coupling reaction speed and efficiency, allowing increased production capacity. In some embodiments, the delivery of energy comprises delivering heat to the reaction chambers. In addition to increasing production capacity, the use of heat allows the use of alternate synthesis chemistries and methods, e.g., the phosphate triester method, which has the advantages of using more stable monomer reagents for synthesis, and of not using tetrazole or its derivatives as condensation catalysts. Heat may be provided by a number of means, including, but not limited to, resistance heaters, visible or infrared light, microwaves, Peltier devices, transfer from fluids or gasses (e.g., via channels or a jacketed system). In some embodiments, heat generated by another component of a synthesis or production facility system (e.g., during a waste neutralization step) is used to provide heat to reaction chambers. In other embodiments, heat is delivered through the use of one or more heated reagents. Delivery of heat to reaction chambers also comprises embodiments wherein heat is created within the reaction chamber, e.g., by magnetic induction or microwave treatment. It is contemplated that heating may be accomplished through a combination of two or more different means.

In some embodiments, the delivery of heat provides substantially uniform heating to two or more reaction chambers. In some embodiments, heating is carried out at a temperature in a range of about 20° C. to about 60° C. The present invention also provides methods for determining an optimum temperature for a particular coupling chemistry. For example, multiple synthesizers are run side-by-side with each machine run at a different temperature. Coupling efficiencies are measured and the optimum temperature for one or more incubations times are determined. In other embodiments, different amounts of heat are delivered to different reaction chambers within a single synthesizer, such that different reaction chemistries or protocols can be run at the same time.

Delivery of heat to a closed system will alter the pressure within the system. It is contemplated that the closed system of the present invention will be configured to tolerate variations in the system pressure (i.e., the pressure within the closed system) related to heating or other energy input to the system. In preferred embodiments, the system (e.g., every component of the system and every junction or seal within the system) will be configured to withstand a range of pressures, e.g., pressures ranging from 0 to at least 1 atm, or about 15 psi. It is contemplated that pressures may be varied between different points within the system. For example, in some embodiments, reagents and waste fluids are moved through the reaction chamber by use of a pressure differential between one end (e.g., an input aperture) and the other (e.g., a drain aperture) of the reaction chamber. In some embodiments, the system of the present invention is configured to use pressure differentials within a pressurized system (e.g., wherein a system segment having lower pressure than another system segment nonetheless has higher pressure than the environment outside the closed system). In some embodiments, the prevention of backward flow of reagents through the system (e.g. in the event of back pressure from a process step such as heating) is controlled by use of pressure. In other embodiments, valves are provided to assist in control of the direction of flow.

In other preferred embodiments, the synthesizer comprises a mixing component configured to mix reaction components, e.g., to facilitate the penetration of reagents into the pores of the solid support. Mixing may be accomplished by a number of means. In some embodiments, mixing is accomplished by forced movement of the fluid through the matrix (e.g., moving it back and forth or circulating it through the matrix using pressure and/or vacuum, or with a fluid oscillator). Mixing may also be accomplished by agitating the contents of the reaction chamber (e.g., stirring, shaking, continuous or pulsed ultra or subsonic waves). In some preferred embodiments, an agitator is used that avoids the creation of standing waves in the reaction mixture. In some preferred embodiments, the agitator is configured to utilize a reaction vessel surface or reaction support surface (e.g., a surface of a synthesis column) to serve as resonant members to transfer energy into fluid within a reaction mixture. In some embodiments, the matrix is an active component of the mixing system. For example, in some embodiments, the matrix comprises paramagnetic particles that may be moved through the use of magnets to facilitate mixing. In some embodiments, the matrix is an active component of both mixing and heating systems (e.g., paramagnetic particles may be agitated by magnetic control and heated by magnetic induction). It is contemplated that any of these mixing means may be used as the sole means of mixing, or that these mixing components may be used in combination, either simultaneously or in sequence. In preferred embodiments, the heating component and the mixing component are under automated control.

In preferred embodiments, a central control processor is used to automate one or more of the synthesis steps or synthesizer operations. The central control processor may also be configured to interact with one or more other components of a production facility (See, Section II, below). In some embodiments, the central control processor regulates valves, controlling the timing, volume, a rate of reagent delivery to the reaction chambers. In preferred embodiments, all delivered reagents are controllable for volume within prescribed ranges at each step of the synthesis process within a protocol independent of other steps.

The present invention is not limited by the range of flow rate used for reagent delivery. However, in preferred embodiments, flow rates are 300-500 μL/sec for all reagents.

Table 1, below, provides an example of reagent delivery times (in seconds) and amounts (in microliters) for a single synthesis cycle. Conditions are provided for four different synthesis scales.

TABLE 1

| Step | 40 nM scale | 200 nM scale | 1 μM scale | 4 μM scale | Time (sec) |
|---|---|---|---|---|---|
| add acetonitrile | 50 | 150 | 250 | 1000 | 0.5 |
| argon purge | | | | | 1 |
| add deblock | 50 | 150 | 250 | 1000 | 0.5 |
| argon purge | | | | | 1 |
| add deblock | 50 | 150 | 250 | 1000 | 0.5 |
| argon purge | | | | | 1 |
| add deblock | 50 | 150 | 250 | 1000 | 0.5 |
| argon purge | | | | | 1 |
| add deblock | 50 | 150 | 250 | 1000 | 0.5 |
| argon purge | | | | | 1 |
| add acetonitrile | 50 | 150 | 250 | 1000 | 0.5 |
| argon purge | | | | | 1 |
| add amidite and | 15 | 30 | 75 | 300 | 30 × 4 |
| tetrazole | 20 | 45 | 115 | 460 | |
| argon purge | | | | | 1 |
| add cap a | 15 | 30 | 60 | 180 | 1 |
| add cap b | 15 | 30 | 60 | 180 | |
| argon purge | | | | | 1 |
| add oxidizer | 40 | 80 | 180 | 360 | 0.5 |
| argon purge | | | | | 1 |
| add acetonitrile | 100 | 200 | 250 | 1000 | |
| argon purge | | | | | |

In preferred embodiments, with the exception of the amidite coupling step, reaction or wash times are controlled by fluid application rate without additional dwell time prior to purging. This is in contrast to methods used with current commercial synthesizers (e.g., 3900 DNA Synthesizers).

A number of different configurations of the synthesizers of the present invention are provided below with exemplary capacities provided. The present invention is not limited to these specific configurations.

A. Pure Batch, Fully Dedicated Fluidics

Batch size is preferably 96 arrayed reaction chambers in a standard microtiter footprint. Synthesis columns could be either independently filled and inserted into a rack to form the array or, preferably, molded in an arrayed format and filled as a batch. If the latter, then all columns should be of a similar type and synthesis operations are grouped accordingly. Column plates are loaded one at a time and replaced at the end of the synthesis process. In some embodiments, loading and unloading is manual—no transport mechanisms required. In other embodiments, loading and unloading is controlled robotically. Fluid connections from the system to the column tray is either established by the system (moving mechanism) or by the user en mass (fixed dispense). Application of reagents is accomplished by a fixed set of multifunctional reagent dispensers, each incorporating all required reagents: each column has a dedicated multiplexed supply line and no motion devices or fluid connection make/break cycles are required. This approach requires a large number of valves (approximately 1000) and is therefore preferably uses very compact, relatively inexpensive and relatively high reliability valves.

| | |
|---|---|
| Estimated walk away time: | 35 minutes |
| Optimal output per day: | approximately 2496 40-mers |
| Valve count: | 1000 |
| Mechanism level: | none |
| Size: | smallest |

B. Pure Batch: Non-dedicated Fluidics

This system is similar to the pure batch system, but rather than dedicated fluidics for each channel, moving reagent dispense heads are provided. This reduces the valve count but adds mechanism. Also, output per day drops in some scale to the valve reduction. A system with approximately 200 valves would produce about 1056 oligonucleotides/2 shift day. Adding a parallel processing station to achieve 2112/day is an option. Walk away time goes up to approximately 80 minutes.

| | |
|---|---|
| Estimated walkaway time: | 1.3 hours |
| Optimal output per day: | approximately 2112 40-mers |
| Valve count: | 400 |
| Mechanisms level: | moderate |
| Size: | moderate |

C. Modified Batch:

This system is similar in configuration to the non-dedicated fluidics batch system described above, but allows multiple plate positions with the system. Walkaway time improves linearly with the number of plates allowed, throughput and other comments are similar. At increasing levels of resident plates, parallel (400 valve system) with 4 plates resident for each parallel line would allow walk away time of 5 hours. In principle, 4 runs of 8 plates could be completed per day producing 3072 oligonucleotides. A 200-valve system configured similarly could produce 1536.

| | |
|---|---|
| Estimated walkaway time: | 5 hours |
| Optimal output per day: | approximately 1536 40-mers |
| Valve count: | 200 |
| Mechanism level: | moderate |
| Size: | moderate |

D. Continuous Batch:

This system is similar to the above system with the addition of queues for feeding plates and accumulating completed plates. The system requires similar fluid handling but adds plate transport mechanisms. The waste system is more complicated due to plate movement. This system allows direct integration to downstream cleave and deprotect system and allows direct integration to synthesis column packing upstream. Throughput is slightly higher than the modified batch system.

| | |
|---|---|
| Estimated walkaway time: | Limited only by onboard storage |
| Optimal output per day: | approximately 1536 40-mers |
| Valve count: | 200 |
| Mechanism level: | high |
| Size: | large |

E. Continuous Parallel:

Rather than a 96-well format, the columns are prepared and presented in strips of 12 columns. The strips are fed through multiple parallel reagent delivery ports. This approach allows greater spacing between adjacent fluidic elements and allows processing of multiple different column types simultaneously. An additional benefit is the likelihood that a closer approach to the theoretical maximum throughput should be routinely achieved. In this embodiment, throughput per valve would be similar to continuous batch, but tubing of throughput is easier.

| | |
|---|---|
| Estimated walkaway time: | limited only by onboard storage |
| Optimal output per day: | approximately 1536 40-mers |
| Valve count: | 200 |
| Mechanism level: | high |
| Size: | large |

(All valve counts are approximate and assume 2 way valves: with multi-position valves, the counts drop accordingly. Also, some rejection may be possible by ganging operations less critically dependent on precise fluid delivery (washes etc). All throughputs assume a nominal cycle for 1 uM scale. Larger scale(s) would be significantly longer. Smaller scales would be essentially similar. Mixing longer and shorter oligonucleotides will drive throughputs to that presented by the longer oligonucleotides).

The synthesizers of the present invention also provide components to reduce or eliminate undesired emissions. A problem with currently available synthesizers is the emission of undesirable gaseous or liquid materials that pose health, environmental, and explosive hazards. Such emissions result from both the normal operation of the instrument and from instrument failures. Emissions that result from instrument failures cause a reduction or loss of synthesis efficiency and can provoke further failures and/or complete synthesizer failure. Correction of failures may require taking the synthesizer off-line for cleaning and repair. The present invention provides nucleic acid synthesizers with components that reduce or eliminate unwanted emissions and that compensate for and facilitate the removal of unwanted emissions, to the extent that they occur at all. The present invention also provides waste handling systems to eliminate or reduce exposure of emissions to the users or the environment. Such systems find use with individual synthesizers, as well as in large-scale synthesis facilities comprising many synthesizers (e.g. arrays of synthesizers).

Whether a system used is open or closed, oligonucleotide synthesis involves the use of an array of hazardous materials, including but not limited to methylene chloride, pyridine, acetic anhydride, 2,6-lutidine, acetonitrile, tetrahydrofurane, and toluene. These reagents can have a variety of harmful effects on those who may be exposed to them. They can be mildly or extremely irritating or toxic upon short-term exposure; several are more severely toxic and/or carcinogenic with long-term exposure. Many can create a fire or explosion hazard if not properly contained. In addition, many of these chemicals must be assessed for emissions from normal operations, e.g for determining compliance with OSHA or environmental agency standards. Malfunction of a system, e.g., as recited above, increases such emissions, thereby increasing the risk of operator exposure, and increasing the risk that an instrument may need to be shut down until risk to an operator is reduced and until any regulatory requirements for operation are met.

Emission or leakage of reagents during operation can have consequences beyond risks to personnel and to the environment. As noted above, instruments may need to be removed from operation for cleaning, leading to a temporary decrease in production capacity of a synthesis facility. Further, any emission or leakage may cause damage to parts of the instrument or to other instruments or aspects of the facility, necessitating repair or replacement of any such parts or aspects, increasing the time and cost of bringing an instrument back into operation. Failure to address emissions or leakage concerns may lead to additional expenses for operation of a facility, e.g., costs for increased or improved fire or explosion containment measures, and addition of costs associated with the elimination of any instrument systems or wiring that have not been determined to be safe for use in such hazardous locations (e.g., by reference to controlling codes, such as electrical codes, or codes covering operations in the presence of flammable and combustible liquids).

The synthesizers of the present invention provide a number of novel features that dramatically improve synthesizer performance and safety compared to available synthesizers. These novel features work both independently and in conjunction to provide enhanced performance. For example, the present invention reduces exposure by improving collection and disposal of emissions that occur during the normal operation of various synthesis instruments. In another embodiment, the present invention reduces exposure by improving aspects of the instrument to reduce risk of malfunctions leading to reagent escape from the system, e.g., through leakage, overflow or other spillage.

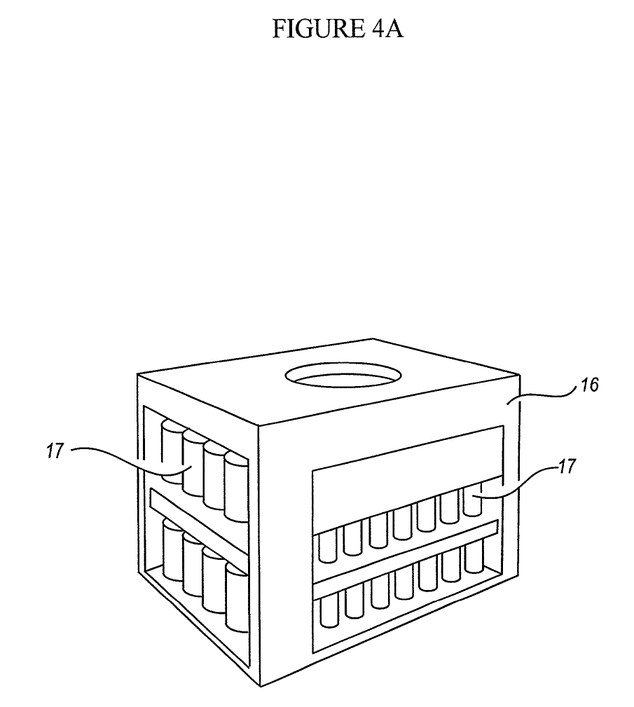

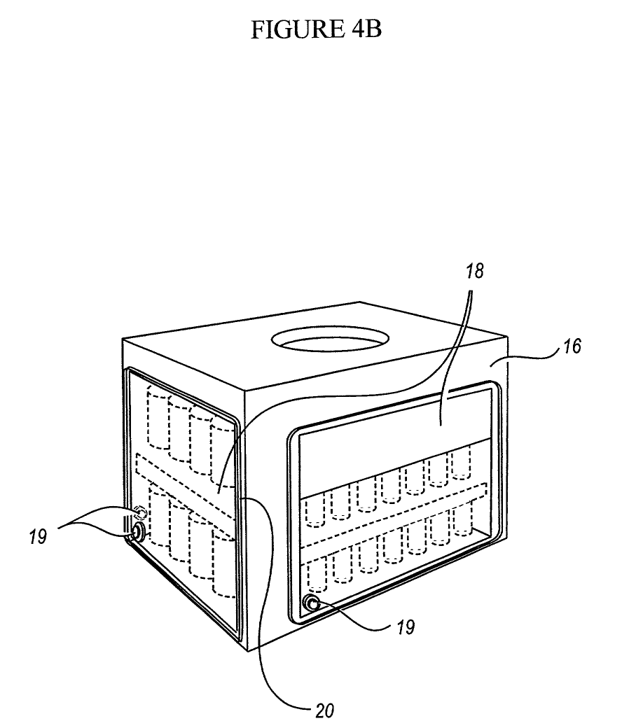

For example, in some embodiments, the present invention provides a means of collecting emissions from the interior of synthesizers by providing a reagent dispensing station. In one embodiment, the reagent dispensing station is an integral part of the base 16 of the synthesizer, as illustrated in FIGS. 4A and 4B. In some embodiments, the reagent dispensing station provides an enclosure for collecting emitted gasses. In some embodiments, the enclosure is created by the provision of a panel 18 to enclose a portion of base 16 containing reagent reservoirs 17, as illustrated in FIG. 4B. In some embodiments, the panel 18 is movable for easy access to reagent reservoirs. In some embodiments, it is removeably attached. Removable attachment may be accomplished by any suitable means, such as through the use of VELCRO, screws, bolts, pins, magnets, temporary adhesives, and the like. In preferred embodiments, at least a portion of the panel 18 is slidably moveable. In preferred embodiments, at least a portion of panel 18 is transparent. In some embodiments, the enclosure of the reagent dispensing station comprises a viewing window that is not in a panel 18.

In some embodiments, the enclosure comprises ventilation tubing. In preferred embodiments, panel 18 comprises a ventilation port 19, e.g., for attachment to ventilation tubing. Since reagent vapors are typically heavier than air, in preferred embodiments, the ventilation tubing is attached at the bottom for the enclosure. In a particularly preferred embodiment, the ventilation port is positioned toward the rear of the instrument.

In some embodiments, the enclosure further comprises an air inlet. In a preferred embodiment, a clearance 20 between the panel 18 and the base 16 provides an air inlet. In a particularly preferred embodiment, the air inlet is positioned toward the front of the instrument. The location of the ventilation port 19 and air inlet is not limited to the panel 18. For example, in an alternative embodiment, the reagent dispensing station comprises a stand for holding the reagent bottles and ventilation tubing, wherein the stand holds the reagent reservoirs and the ventilation tubing removes emitted gases.

Ventilation may be continuous or under the control of an operator. For example, in some embodiments, when the panel 18 is in a closed position, ventilation occurs continuously through the ventilation port 19 or at regular intervals. In other embodiments, an operator may manually activate ventilation prior to opening the panel 18. In still other embodiments, ventilation occurs in an automated fashion immediately prior to the opening of panel 18. For example, where the opening of panel 18 is controlled by a computer processor, activation of the “open” routine triggers ventilation prior to the physical opening of panel 18. In still other embodiments, the contents of the reagent containers are monitored by a sensor and the ventilation is triggered when one or more of the reagent containers are depleted. In some embodiments, the panel 18 is also automatically open, indicating the need for additional reagents and/or allowing an automated reagent container delivery system to supply reagents to the system.

II. Production Facility

The present invention provides synthesizer arrays (e.g., groups of synthesizers). In some embodiments, the synthesizers are arranged in banks. For example, a given bank of synthesizers may be used to produce one set of oligonucleotides. The present invention is not limited to any one synthesizer. Indeed, a variety of synthesizers are contemplated, including, but not limited to the synthesizers of the present invention, MOSS EXPEDITE 16-channel DNA synthesizers (PE Biosystems, Foster City, Calif.), OligoPilot (Amersham Pharmacia,), and the 3900 and 3948 48-Channel DNA synthesizers (PE Biosystems, Foster City, Calif.). In some embodiments, synthesizers are modified or are wholly fabricated to meet physical or performance specifications particularly preferred for use in the synthesis component of the present invention. In some embodiments, two or more different DNA synthesizers are combined in one bank in order to optimize the quantities of different oligonucleotides needed. This allows for the rapid synthesis (e.g., in less than 4 hours) of an entire set of oligonucleotides (all the oligonucleotide components needed for a particular assay, e.g., for detection of one SNP using an INVADER assay [Third Wave Technologies, Madison, Wis.]).

In some embodiments the DNA synthesizer component includes at least 100 synthesizers. In other embodiments, the DNA synthesizer component includes at least 200 synthesizers. In still other embodiments, the DNA synthesizer component includes at least 250 synthesizers. In some embodiments, the DNA synthesizers are run 24 hours a day.

A. Automated and Fail-Safe Reagent Supply

In some embodiments, the DNA synthesizers in the oligonucleotide synthesis component further comprise an automated reagent supply system. The automated reagent supply system delivers reagents necessary for synthesis to the synthesizers from a central supply area. In some embodiments, the central supply area is provided in an isolated room equipped for accommodating leakage, fires, and explosions without threatening other portions of the synthesis facility, the environment, or humans. Where the central supply area provides reagents for multiple synthesizers, in some embodiments, the system is configured to allow banks of synthesizer or individual synthesizer to be removed from the system (e.g., for maintenance or repair) without interrupting activity at other synthesizers. Thus, the present invention provides an efficient fail-safe reagent delivery system.

For example, in some embodiments, acetonitrile is supplied via tubing (e.g., stainless steel tubing) through the automated supply system. De-blocking solution may also be supplied directly to DNA synthesizers through tubing. In some preferred embodiments, the reagent supply system tubing is designed to connect directly to the DNA synthesizers without modifying the synthesizers. Additionally, in some embodiments, the central reagent supply is designed to deliver reagents at a constant and controlled pressure. The amount of reagent circulating in the central supply loop is maintained at 8 to 12 times the level needed for synthesis in order to allow standardized pressure at each instrument. The excess reagent also allows new reagent to be added to the system without shutting down. In addition, the excess of reagent allows different types of pressurized reagent containers to be attached to one system. The excess of reagents in one centralized system further allows for one central system for chemical spills and fire suppression.

In some embodiments, the DNA synthesis component includes a centralized argon delivery system. The system includes high-pressure argon tanks adjacent to each bank of synthesizers. These tanks are connected to large, main argon tanks for backup. In some embodiments, the main tanks are run in series. In other embodiments, the main tanks are set up in banks. In some embodiments, the system further includes an automated tank switching system. In some preferred embodiments, the argon delivery system further comprises a tertiary backup system to provide argon in the case of failure of the primary and backup systems.

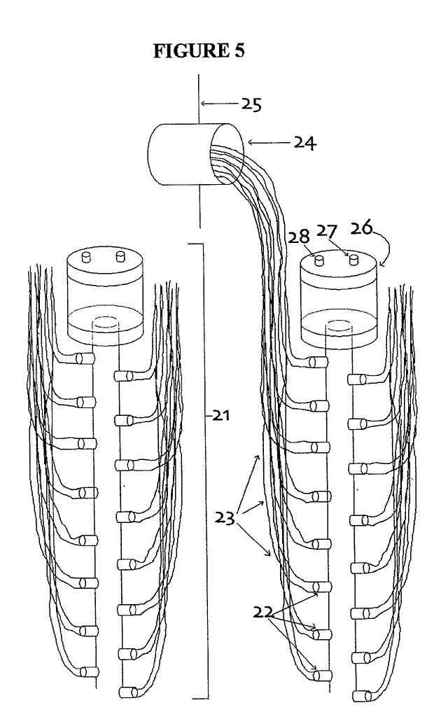

In some embodiments, one or more branched delivery components are used between the reagent tanks and the individual synthesizers or banks of synthesizers. For example, in some embodiments, acetonitrile is delivered through a branched metal structure (e.g., the structure described in FIG. 5). Where more than one branched delivery component is used, in preferred embodiments, each branched delivery component is individually pressurized.

The present invention is not limited by the number of branches in the branched delivery component. In preferred embodiments, each branched delivery component (21) contains ten or more branches (22). Reagent tanks may be connected to the branched delivery components using any number of configurations. For example, in some embodiments, a single reagent tank is matched with a single branched component. In other embodiments, a plurality of reagent tanks is used to supply reagents to one or more branched components. In some such embodiments, the plurality of tanks may be attached to the branched components through a single feed line, wherein one or a subset of the tanks feeds the branched components until empty (or substantially empty), whereby a second tank or subset of tanks is accessed to maintain a continuous supply of reagent to the one or more branched components. To automate the monitoring and switching of tanks, an ultrasonic level sensor may be applied.

In some embodiments, each branch of the branched delivery component provides reagent to one synthesizer or to a bank of synthesizers through connecting tubing (23). In preferred embodiments, tubing is continuous (i.e., provides a direct connection between the delivery branch and the synthesizer). In some preferred embodiments, the tubing comprises an interior diameter of 0.25 inches or less (e.g., 0.125 inches). In some embodiments, each branch contains one or more valves (preferably one). While the valve may be located at any position along the delivery line, in preferred embodiments, the valve is located in close proximity to the synthesizer. In other embodiments, reagent is provided directly to synthesizers without any joints or valves between the branched delivery component and the synthesizers.

In some embodiments, the solvent is contained in a cabinet designed for the safe storage of flammable chemicals (a “flammables cabinet”) and the branched structure is located outside of the cabinet and is fed by the solvent container through tubing passed through the wall of the cabinet. In other embodiments, the reagent and branched system is stored in an explosion proof room or chamber and the solvent is pumped via tubing through the wall of the explosion proof room. In preferred embodiments, all of the tubing from each of the branches is fed through the wall in at a single location (e.g., through a single hole (24) in the wall (25)).

The reagent delivery system of the present invention provides several advantages. For example, such a system allows each synthesizer to be turned off (e.g., for servicing) independent of the other synthesizers. Use of continuous tubing reduces the number of joints and couplings, the areas most vulnerable to failure, between the reagent sources and the synthesizers, thereby reducing the potential for leakage or blockage in the system. Use of continuous tubing through inaccessible or difficult-to-access areas reduces the likelihood that repairs or service will be needed in such areas. In addition, fewer valves results in cost savings.

In some embodiments, the branched tubing structure further provides a sight glass (26). In preferred embodiments, the sight glass is located at the top of the branched delivery structure. The sight glass provides the opportunity for visual and physical sampling of the reagent. For example, in some embodiments, the sight glass includes a sampling valve (27) (e.g., to collect samples for quality control). In some embodiments, the site glass serves as a trap for gas bubbles, to prevent bubbles from entering the connecting tubing (23). In other embodiments, the sight glass contains a vent (e.g., a solenoid valve) for de-gassing of the system (28). In some embodiments, scanning of the sight glass (e.g., spectrophotometrically) and sampling are automated. The automated system provides quality control and feedback (e.g., the presence of contamination).

In other embodiments, the present invention provides a portable reagent delivery system. In some embodiments, the portable reagent delivery system comprises a branched structure connected to solvent tanks that are contained in a flammables cabinet. In preferred embodiments, one reagent delivery system is able to provide sufficient reagent for 40 or more synthesizers. These portable reagent delivery systems of the present invention facilitate the operation of mobile (portable) synthesis facilities. In another embodiment, these portable reagent delivery systems facilitate the operation of flexible synthesis facilities that can be easily re-configured to meet particular needs of individual synthesis projects or contracts. In some embodiments, a synthesis facility comprises multiple portable reagent delivery systems.

B. Waste Collection

In some embodiments, the DNA synthesis component further comprises a centralized waste collection system. The centralized waste collection system comprises cache pots for central waste collection. In some embodiments, the cache pots include level detectors such that when waste level reaches a preset value, a pump is activated to drain the cache into a central collection reservoir. In preferred embodiments, ductwork is provided to gather fumes from cache pots. The fumes are then vented safely through the roof, avoiding exposure of personnel to harmful fumes. In preferred embodiments, the air handling system provides an adequate amount of air exchange per person to ensure that personnel are not exposed to harmful fumes. The coordinated reagent delivery and waste removal systems increase the safety and health of workers, as well as improving cost savings.

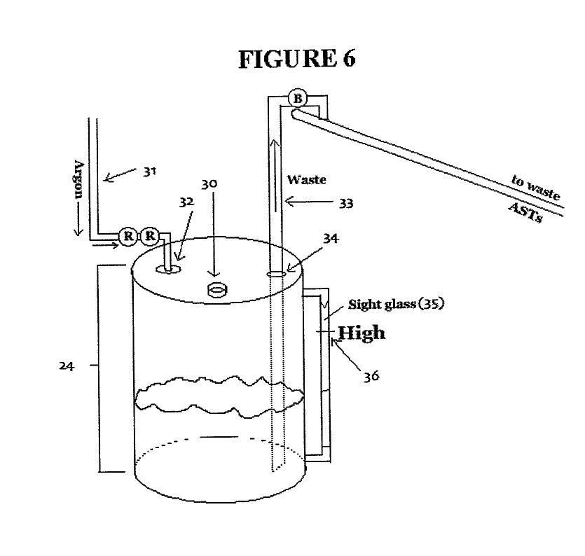

In some embodiments, the solvent waste disposal system comprises a waste transfer system. In some preferred embodiments, the system contains no electronic components. In some preferred embodiments, the system comprises no moving parts. For example, in some embodiments, waste is first collected in a liquid transfer drum (29) designed for the safe storage of flammable waste (See FIG. 6 for an exemplary waste disposal system). In some embodiments, waste is manually poured into the drum through a waste channel (30). In preferred embodiments, solvent waste is automatically transported (e.g., through tubing) directly from synthesizers to the drum (29). To drain the liquid transfer drum (29), argon is pumped from a pressurized gas line (31) into the drum through a first opening (32), forcing solvent waste out an output channel (33) at a second opening (34) (e.g., through tubing) into a centralized waste collection area. In preferred embodiments, the argon is pumped at low pressure (e.g., 3-10 pounds per square inch (psi), preferably 5 psi or less). In some embodiments, the drum (29) contains a sight glass (35) to visualize the solvent level. In some embodiments, the level is visualized manually and the disposal system is activated when the drum (29) has reached a selected threshold level (36). In other embodiments, the level is automatically detected and the disposal system is automatically activated when the drum (29) has reached the threshold level (36).

The solvent waste transfer system of the present invention provides several advantages over manual collection and complex systems. The solvent waste system of the present invention is intrinsically safe, as it can be designed with no moving or electrical parts. For example, the system described above is suitable for use in Division I/Class I space under EPA regulations.

Some process steps may put out caustic waste. For example, deprotection of synthesized oligonucleotides generally includes treatment with NH₄OH. In some embodiments, caustic waste is neutralized before disposal, e.g., to a sanitary sewer. In preferred embodiments, the neutralization of the waste is checked (e.g., by measurement of pH) to ensure that it is in an appropriate condition for disposal via the intended system (e.g., the sanitary sewer system).

In some embodiments, waste from each deprotection station is neutralized before collection to a centralized waste collection or disposal system. In other embodiments, caustic waste from a plurality of deprotection stations is collected before neutralization.

By way of example, and not intended as a limitation, the following provides a description for one embodiment of a centralized collection and neutralization system for caustic waste. The system may comprise collection of caustic waste from one or more stations in a tank, e.g., a carboy. In some embodiments, the amount of neutralizing reagent required to neutralize a defined amount of caustic waste is calculated, based on the volume and content of the waste. In some embodiments, the calculated amount of neutralizing reagent is added after collection of the waste. In preferred embodiments, the calculated amount of neutralizing reagent is provided in the carboy, such that when the carboy is full or when the combined volume of the neutralizer and waste reaches a predetermined volume, the waste has been neutralized.

In one embodiment, the carboy is provided with a pH probe for measurement of the pH of the collected waste. In some embodiments, the system provides a means of altering the pH of the collected waste. In preferred embodiments, the altering of the pH occurs in response to a measured pH value for the collected waste. For example, if the pH is determined to be outside a certain range, (e.g. if it does not fall between, for example, pH 7 and pH 9), the system provides a reagent selected to adjust the pH to the selected range (e.g., if the pH is found to be high, the system dispenses an acidic solution for neutralization; if the pH is low, the system dispenses a basic solution for neutralization). When the pH comes into the selected range, the system shuts off the dispenser. For the step of dispensing a neutralizing reagent, any system suitable for the controlled delivery of a reagent is contemplated. For example, discharge may be accomplished via a mechanical dispenser, or discharge can be accomplished via non-mechanical means, e.g., via control of air pressure.

In some embodiments, neutralization treatment is provided to the collected waste in bulk, e.g., when the carboy is full or when it reaches a predetermined threshold level. In other embodiments, neutralization is periodic. In some embodiments, periodic neutralization is set to occur at particular times, e.g., at particular times of day, or whenever a particular interval of time has passed since the last treatment. In other embodiments, periodic treatment is set to respond to a condition of the waste container, such as whenever a new addition of waste material occurs, or whenever the pH is not within the selected range. In yet other embodiments, periodic treatment occurs based on a combination of these or other factors.

In a preferred embodiment, the carboy is provided with a means for mixing, such as a stirrer or agitator. In some embodiments, the system comprises a device for keeping a precipitate suspended. In some embodiments, the system provides a filter for removing precipitates, particulates or other non-liquid matter in the collected waste. In other preferred embodiments, the system provides a means of venting gasses. In particularly preferred embodiments, the gasses are collected for disposal through a centralized ventilation system.

C. Centralized Control System

In some embodiments, all of the DNA synthesizers in the synthesis component are attached to a centralized control system. The centralized control system controls all areas of operation, including, but not limited to, power, pressure, reagent delivery, waste, and synthesis. In some preferred embodiments, the centralized control system includes a clean electrical grid with uninterrupted power supply. Such a system minimizes power level fluctuations. In additional preferred embodiments, the centralized control system includes alarms for air flow, status of reagents, and status of waste containers. The alarm system can be monitored from the central control panel. The centralized control system allows additions, deletions, or shutdowns of one synthesizer or one block of synthesizers without disrupting operations of other instruments. The centralized power control allows user to turn instruments off instrument by instrument, bank by bank, or the entire module.

D. Integrated Production Process

In some embodiments, the present invention provides an automated production process. In some embodiments, the automated production process includes an oligonucleotide synthesizer component and an oligonucleotide processing component. In some embodiments, the oligonucleotide production component includes multiple components, including but not limited to, an oligonucleotide cleavage and deprotection component, an oligonucleotide purification component, an oligonucleotide dry down component; an oligonucleotide de-salting component, an oligonucleotide dilute and fill component, and a quality control component. In some embodiments, the automated DNA production process of the present invention further includes automated design software and supporting computer terminals and connections, a product tracking system (e.g., a bar code system), and a centralized packaging component. In some embodiments, the components are combined in an integrated, centrally controlled, automated production system. The present invention thus provides methods of synthesizing several related oligonucleotides (e.g., components of a kit) in a coordinated manner. In some preferred embodiments, a sample holder (e.g., a reaction support) is shared between two or more of the components of the production process. The sample holder may be transferred by hand or robotically from one component to the next.

1. Oligonucleotide Design Component

In some embodiments of the present invention, the DNA production process included an automated oligonucleotide design system. The system includes software utilized to design the sequence of the oligonucleotide. The software and parameters chosen vary according to the application that the oligonucleotides are designed for use in.

For example, in some embodiments where an oligonucleotide is designed for use in the INVADER assay to detect a SNP, the sequence(s) of interest (synthesis request information) are entered into the INVADERCREATOR program (Third Wave Technologies, Madison, Wis.). The program designs probes for both the sense and antisense strand. Strand selection is based upon the ease of synthesis, minimization of secondary structure formation, and manufacturability. In some embodiments, the user chooses the strand for sequences to be designed for. In other embodiments, the software automatically selects the strand. By incorporating thermodynamic parameters for optimum probe cycling and signal generation (Allawi and SantaLucia, Biochemistry, 36:10581 [1997]), oligonucleotide probes are designed to operate at a preselected assay temperature. In particular embodiments, oligonucleotide probes are designed to operate at an assay temperature of 63° C. Based on these criteria, a final probe set (e.g., primary probes for 2 alleles and an INVADER oligonucleotide) is selected.

In some embodiments, the INVADERCREATOR system is a web-based program with secure site access that contains a link to the BLAST search web site at the National Library of Medicine at the NIH, and can be linked to RNAstructure (Mathews et al., RNA 5:1458 [1999]), a software program that incorporates mfold (Zuker, Science, 244:48 [1989]). RNAstructure tests the proposed oligonucleotide designs generated by INVADERCREATOR for potential uni- and bimolecular complex formation. INVADERCREATOR is open database connectivity (ODBC)-compliant and uses the Oracle database for export/integration. The INVADERCREATOR system was configured with Oracle to work well with UNIX systems, as most genome centers are UNIX-based.

The INVADERCREATOR analysis is provided on a separate Sun server so it can handle analysis of large batch jobs. For example, a customer can submit up to 2,000 SNP sequences in one e-mail. The server passes the batch of sequences on to the INVADERCREATOR software, and, when initiated, the program designs SNP sets. Probe set designs are returned to the user within 24 hours of receipt of the sequences.

Each INVADER reaction includes at least two target sequence-specific oligonucleotides for the primary reaction: an upstream INVADER oligonucleotide and a downstream Probe oligonucleotide. Generally, these oligonucleotides are unlabeled. The INVADER oligonucleotide is designed to bind stably at the reaction temperature, while the probe is designed to freely associate and disassociate with the target strand, with cleavage occurring only when an uncut probe hybridizes to a target adjacent to an overlapping INVADER oligonucleotide. In some embodiments, the probe includes a 5′ flap that is not complementary to the target, and this flap is released from the probe when cleavage occurs. In some embodiments, the released flap participates as an INVADER oligonucleotide in a secondary reaction.

To select a probe sequence that will perform optimally at a pre-selected reaction temperature, the melting temperature (TM) of the SNP to be detected is calculated using the nearest-neighbor model and published parameters for DNA duplex formation (Allawi and SantaLucia, Biochemistry, 36:10581 [1997]. Because the assay's salt concentrations are often different than the solution conditions in which the nearest-neighbor parameters were obtained (1M NaCl and no divalent metals), and because the presence and concentration of the enzyme influences the optimal reaction temperature, an adjustment is generally made to the calculated TM to determine the optimal temperature at which to perform a reaction. One way of compensating for these factors is to vary the value provided for the salt concentration within the melting temperature calculations. This adjustment is termed a ‘salt correction’. As used herein, the term “salt correction” refers to a variation made in the value provided for a salt concentration for the purpose of reflecting the effect on a TM calculation for a nucleic acid duplex of a non-salt parameter or condition affecting said duplex. Variation of the values provided for the strand concentrations will also affect the outcome of these calculations. By using a value of 0.5 M NaCl (SantaLucia, Proc Natl Acad Sci USA, 95:1460 [1998]) and strand concentrations of about 1 mM of the probe and 1 fM target, the algorithm used for calculating probe-target melting temperature has been adapted for use in predicting optimal INVADER assay reaction temperature. For a set of 30 probes, the average deviation between optimal assay temperatures calculated by this method and those experimentally determined is about 1.5° C.

The length of the downstream probe to a given SNP is defined by the temperature selected for running the reaction (e.g., 63° C.). Starting from the position of the variant nucleotide on the target DNA (the target base that is paired to the probe nucleotide 5′ of the intended cleavage site), an iterative procedure is used by which the length of the SNP region is increased by one base pair until a calculated optimal reaction temperature (TM plus salt correction to compensate for enzyme effect) matching the pre-selected, desired reaction temperature is reached. The non-complementary arm of the probe is preferably selected to allow the secondary reaction to cycle at the same reaction temperature, and is screened using programs such as mfold (Zuker, Science, 244: 48 [1989]) or Oligo 5.0 (Rychlik and Rhoads, Nucleic Acids Res, 17: 8543 [1989]) for the possible formation of dimer complexes or secondary structures that could interfere with the reaction. The same principles are also followed for INVADER oligonucleotide design. Briefly, starting from the position N on the target DNA, the 3′ end of the INVADER oligonucleotide is designed to have a nucleotide not complementary to either allele suspected of being contained in the sample to be tested. The mismatch does not adversely affect cleavage (Lyamichev et al., Nature Biotechnology, 17: 292 [1999]), and it can enhance probe cycling, presumably by minimizing coaxial stabilization effects between the two probes. Additional residues complementary to the target DNA starting from residue N−1 are then added in the upstream direction until the stability of the INVADER oligonucleotide-target hybrid exceeds that of the probe (and therefore the planned assay reaction temperature) by 15-20° C.

It is one aspect of the assay design that the all of the probe sequences may be selected to allow the primary and secondary reactions to occur at the same optimal temperature, so that the reaction steps can run simultaneously. In an alternative embodiment, the probes may be designed to operate at different optimal temperatures, so that the reactions steps are not simultaneously at their temperature optima.

The present invention is not limited to the use of the INVADERCREATOR software. Indeed, a variety of software programs are contemplated and are commercially available, including, but not limited to GCG Wisconsin Package (Genetics computer Group, Madison, Wis.) and Vector NTI (Informax, Rockville, Md.).

2. Oligonucleotide Synthesis Component

Once a particular oligonucleotide sequence or set of sequences has been chosen, sequences are sent (e.g., electronically) to a high-throughput oligonucleotide synthesizer component. In some preferred embodiments, the high-throughput synthesizer component contains multiple DNA synthesizers. Such systems are described in detail above.

3. Oligonucleotide Processing Components

In some embodiments, the automated DNA production process further comprises one or more oligonucleotide production components, including, but not limited to, an oligonucleotide cleavage and deprotection component, an oligonucleotide purification component, a dry-down component, a desalting component, a dilution and fill component, and a quality control component.

A. Oligonucleotide Cleavage and Deprotection

After synthesis is complete, the oligonucleotides are moved to the cleavage and deprotection station. In some embodiments, the transfer of oligonucleotides to this station is automated and controlled by robotic automation. In some embodiments, the entire cleavage and deprotection process is performed by robotic automation. In some embodiments, a deprotecting reagent (e.g., NH₄OH or other deprotecting reagent) is supplied through the automated reagent supply system.

Accordingly, in some embodiments, oligonucleotide deprotection is performed in multi-sample containers (e.g., 96 well covered dishes) in an oven. This method is designed for the high-throughput system of the present invention and is capable of the simultaneous processing of large numbers of samples. This method provides several advantages over the standard method of deprotection in vials. For example, sample handling is reduced (e.g., labeling of vials dispensing of concentrated NH₄OH to individual vials, as well as the associated capping and uncapping of the vials, is eliminated). This reduces the risks of contamination or mislabeling and decreases processing time. Where such methods are used to replace human pipetting of samples and capping of vials, the methods save many labor hours per day. The method also reduces consumable requirements by eliminating the need for vials and pipette tips, reduces equipment needs by eliminating the need for pipettes, and improves worker safety conditions by reducing worker exposure to ammonium hydroxide. The potential for repetitive motion disorders is also reduced. Deprotection in a multi-well plate further has the advantage that the plate can be directly placed on an automated desalting apparatus (e.g., TECAN Robot).

During the development of the present invention, the plate was optimized to be functional and compatible with the deprotection methods. In some embodiments, the plate is designed to be able to hold as much as two milliliters of oligonucleotide and ammonium hydroxide. If deep well plates are used, automated downstream processing steps may need to be altered to ensure that the full volume of sample is extracted from the wells. In some embodiments, the multi-well plates used in the methods of the present invention comprise a tight sealing lid/cover to protect from evaporation, provide for even heating, and are able to withstand temperatures necessary for deprotection. Attempts with initial plates were not successful, having problems with lids that were not suitably sealed and plates that did not withstand deprotection temperatures.

In some embodiments (e.g., processing of target and INVADER oligonucleotides), oligonucleotides are cleaved from the synthesis support in the multi-well plates. In other embodiments (e.g., processing of probe oligonucleotides), oligonucleotides are first cleaved from the synthesis column and then transferred to the plate for deprotection.

B. Oligonucleotide Purification

In some embodiments, following deprotection and cleavage from the solid support, oligonucleotides are further purified. Any suitable purification method may be employed, including, but not limited to, high pressure liquid chromatography (HPLC) (e.g., using reverse phase C18 and ion exchange), reverse phase cartridge purification, and gel electrophoresis. However, in preferred embodiments, purification is carried out using ion exchange HPLC chromatography.

In some embodiments, multiple HPLC instruments are utilized, and integrated into banks (e.g., banks of 8 HPLC instruments). Each bank is referred to as an HPLC module. Each HPLC module consists of an automated injector (e.g., including, but not limited to, Leap Technologies 8-port injector) connected to each bank of automated HPLC instruments (e.g., including, but not limited to, Beckman-Coulter HPLC instruments). The automatic Leap injector can handle four 96-well plates of cleaved and deprotected oligonucleotides at a time. The Leap injector automatically loads a sample onto each of the HPLCs in a given bank. The use of one injector with each bank of HPLC provides the advantage of reducing labor and allowing integrated processing of information.

In some embodiments, oligonucleotides are purified on an ion exchange column using a salt gradient. Any suitable ion exchange functionality or support may be utilized, including but not limited to, Source 15 Q ion exchange resin (Pharmacia). Any suitable salt may be utilized for elution of oligonucleotides from the ion exchange column, including but not limited to, sodium chloride, acetonitrile, and sodium perchlorate. However, in preferred embodiments, a gradient of sodium perchlorate in acetonitrile and sodium acetate is utilized.

In some embodiments, the gradient is run for a sufficient time course to capture a broad range of sizes of oligonucleotides. For example, in some embodiments, the gradient is a 54 minute gradient carried out using the method described in Tables 1 and 2. Table 1 describes the HPLC protocol for the gradient. The time column represents the time of the operation. The module column represents the equipment that controls the operation. The function column represents the function that the HPLC is performing. The value column represents the value of the HPLC function at the time specified in the time column. Table 2 describes the gradient used in HPLC purification. The column temperature is 65° C. Buffer A is 20 mM Sodium Perchlorate, 20 mM Sodium Acetate, 10 Acetonitrile, pH 7.35. Buffer B is 600 mM Sodium Perchlorate, 20 mM Sodium Acetate, 10 Acetonitrile, pH 7.35.

In some embodiments, the gradient is shortened. In preferred embodiments, the gradient is shortened so that a particular gradient range suitable for the elution of a particular oligonucleotide being purified is accomplished in a reduced amount of time. In other preferred embodiments, the gradient is shortened so that a particular gradient range suitable for the elution of any oligonucleotide having a size within a selected size range is accomplished in a reduced amount of time. This latter embodiment provides the advantages that the worker performing HPLC need not have foreknowledge of the size of an oligonucleotide within the selected size range, and the protocol need not be altered for purification of any oligonucleotide having a size within the range.

In a particularly preferred embodiment, the gradient is a 34 minute gradient described in the Tables 4 and 5. The parameters and buffer compositions are as described for Tables 2 and 3. Reducing the gradient to 34 minutes increases the capacity of synthesis per HPLC instrument and reduces buffer usage by 50% compared to the 54 minute protocol described above. The 34 minute HPLC method of the present invention has the further advantage of being optimized to be able to separate oligonucleotides of a length range of 23-39 nucleotides without any changes in the protocol for the different lengths within the range. Previous methods required changes for every 2-3 nucleotide change in length. In yet other embodiments, the gradient time is reduced even further (e.g., to less than 30 minutes, preferably to less than 20 minutes, and even more preferably, to less than 15 minutes). Any suitable method may be utilized that meets the requirements of the present invention (e.g., able to purify a wide range of oligonucleotide lengths using the same protocol).

In some embodiments, separate sets of HPLC conditions, each selected to purify oligonucleotides within a different size range, may be provided (e.g., may be run on separate HPLCs or banks of HPLCs). Thus, in some embodiments of the present invention, a first bank of HPLCs are configured to purify oligonucleotides using a first set of purification conditions (e.g., for 23-39 mers), while second and third banks are used for the shorter and longer oligonucleotides. Use of this system allows for automated purification without the need to change any parameters from purification to purification and decreases the time required for oligonucleotide production.

In some embodiments, the HPLC station is equipped with a central reagent supply system. In some embodiments, the central reagent system includes an automated buffer preparation system. The automated buffer preparation system includes large vat carboys that receive pre-measured reagents and water for centralized buffer preparation. The buffers (e.g., a high salt buffer and a low salt buffer) are piped through a circulation loop directly from the central preparation area to the HPLCs. In some embodiments, the conductivity of the solution in the circulation loop is monitored to verify correct content and adequate mixing. In addition, in some embodiments, circulation lines are fitted with venturis for static mixing of the solutions as they are circulated through the piping loop. In still further embodiments, the circulation lines are fitted with 0.05 μm filters for sterilization. In some preferred embodiments, the buffer tanks contain from about 100 liters to about 500 liters of buffer. The use of large buffer tanks allows for a more consistent buffer mixture. In some preferred embodiments, the individual buffer systems are supported by a high purity water purification system so as to avoid having to purchase individual containers.

In some preferred embodiments, the HPLC purification step is carried out in a clean room environment. The clean room includes a HEPA filtration system. All personnel in the clean room are outfitted with protective gloves, hair coverings, and foot coverings.

In preferred embodiments, the automated buffer prep system is located in a non-clean room environment and the prepared buffer is piped through the wall into the clean room.

Each purified oligonucleotide is collected into a tube (e.g., a 50-ml conical tube) in a carrying case in the fraction collector. Collection is based on a set method, which is triggered by an absorbance rate change within a predetermined time window. In some embodiments, the method uses a flow rate of 5 ml/min (the maximum rate of the pumps is 10 ml/min.) and each column is automatically washed before the injector loads the next sample.

(Det=detector; %B=percent of buffer B; flow rate values in ml/min)

TABLE 2
54 Minute HPLC Method

| Time (min) | Module | Function | Value | Duration (min) |
|---|---|---|---|---|
| 0 | Pump | % B | 22.00 | 4.0 |
| 0 | Det 166-3 | Autozero ON | | |
| 0 | Det 166-3 | Relay ON | 3.0 | 0.10 |
| 4 | Pump | % B | 37.00 | 43.00 |
| 47 | Pump | % B | 100.00 | 0.50 |
| 47.5 | Pump | Flow Rate | 7.5 | 0.00 |
| 50.0 | Pump | % B | 5.0 | 0.50 |
| 53.45 | Det 166-3 | Stop Data | | |

TABLE 3
54 Minute HPLC Method

| | | |
|---|---|---|
| Time | Gradient | Flow Rate |

| | | | |
|---|---|---|---|
| 0 | | 5% B/95% A | 5 ml/min |
| 0-4 | min | 5-22% B | 5 ml/min |
| 4-47 | min | 22-37% B | 5 ml/min |
| 47-47.5 | min | 37-100% B | 7.5 ml/min |
| 47.5-50 | min | 100% B | 7.5 ml/min |
| 50-50.5 | min | 100-5% B | 7.5 ml/min |
| 50.5-53.5 | min | 5% B | 7.5 ml/min |

TABLE 4
34 Minute HPLC Method

| Time (min) | Module | Function | Value | Duration |
|---|---|---|---|---|
| 0 | Pump | % B | 26.00 | 2.0 |
| 0 | Det 166-3 | Autozero ON | | |
| 0 | Det 166-3 | Relay ON | 3.0 | 0.10 |
| 2 | Pump | % B | 36.00 | 27.00 |
| 29 | Pump | % B | 100.00 | 0.50 |
| 29.5 | Pump | Flow Rate | 7.5 | 0.00 |
| 32 | Pump | % B | 5.0 | 0.50 |
| 33.45 | Det 166-3 | Stop Data | | |

TABLE 5
34 Minute HPLC Method

| | | |
|---|---|---|
| Time | Gradient | Flow Rate |

| | | | |
|---|---|---|---|
| 0 | | 5% B/95% A | 5 ml/min |
| 0-2 | min | 5-26% B | 5 ml/min |
| 2-29 | min | 26-36% B | 5 ml/min |
| 29-29.5 | min | 36-100% B | 6.5 ml/min |
| 29.5-32 | min | 100% B | 7.5 ml/min |
| 32-32.5 | min | 100-5% B | 7.5 ml/min |
| 32.5-33.5 | min | 5% B | 7.5 ml/min |

C. Dry-Down Component

When the fraction collector is full of eluted oligonucleotides, they are transferred (e.g., by automated robotics or by hand) to a drying station. For example, in some embodiments, the samples are transferred to customized racks for Genevac centrifugal evaporator to be dried down. In preferred embodiments, the Genevac evaporator is equipped with racks designed to be used in both the Genevac and the subsequent desalting step. The Genevac evaporator decreases drying time, relative to other commercially available evaporators, by 60%.

D. Desalting Component

In some embodiments, following HPLC, oligonucleotides are desalted. In other embodiments, oligonucleotides are not HPLC purified, but instead proceed directly from deprotection to desalting. In some embodiments, the desalting stations have TECAN robot systems for automated desalting. The system employs a rack that has been designed to fit the TECAN robot and the Genevac centrifugal evaporator without transfer to a different rack or holder. The racks are designed to hold the different sizes of desalting columns, such as the NAP-5 and NAP-10 columns. The TECAN robot loads each oligonucleotide onto an individual NAP-5 or NAP-10 column, supplies the buffer, and collects the eluate. If desired, desalted oligonucleotides may be frozen or dried down at this point.

In some embodiments, following desalting, INVADER and target oligonucleotides are analyzed by mass spectroscopy. For example, in some embodiments, a small sample from the desalted oligonucleotide sample is removed (e.g., by a TECAN robot) and spotted on an analysis plate, which is then placed into a mass spectrometer. The results are analyzed and processed by a software routine. Following the analysis, failed oligonucleotides are automatically reordered, while oligonucleotides that pass the analysis are transported to the next processing step. This preliminary quality control analysis removes failed oligonucleotides earlier in the processing, thus resulting in cost savings and improving cycle times.

E. Oligonucleotide Dilution and Fill Component

In some embodiments, the oligonucleotide production process further includes a dilute and fill module. In some embodiments, each module consists of three automated oligonucleotide dilution and normalization stations. Each station consists of a network-linked computer and an automated robotic system (e.g., including but not limited to Biomek 2000). In one embodiment, the pipetting station is physically integrated with a spectrophotometer to allow machine handling of every step in the process. All manipulations are carried out in a HEPA-filtered environment. Dissolved oligonucleotides are loaded onto the Biomek 2000 deck the sequence files are transferred into the Biomek 2000. The Biomek 2000 automatically transfers a sample of each oligonucleotide to an optical plate, which the spectrophotometer reads to measure the A260 absorbance. Once the A260 has been determined, an Excel program integrated with the Biomek software uses absorbance and the sequence information to prepare a dilution table for each oligonucleotide. The Biomek employs that dilution table to dilute each oligonucleotide appropriately. The instrument then dispenses oligonucleotides into an appropriate vessel (e.g., 1.5 ml microtubes).

In some preferred embodiments, the automated dilution and fill system is able to dilute different components of a kit (e.g., INVADER and probe oligonucleotides) to different concentrations. In other preferred embodiments, the automated dilution and fill module is able to dilute different components to different concentrations specified by the end user.

F. Quality Control Component

In some embodiments, oligonucleotides undergo a quality control assay before distribution to the user. The specific quality control assay chosen depends on the final use of the oligonucleotides. For example, if the oligonucleotides are to be used in an INVADER SNP detection assay, they are tested in the assay before distribution.

In some embodiments, each SNP set is tested in a quality control assay utilizing the Beckman Coulter SAGIAN CORE System. In some embodiments, the results are read on a real-time instrument (e.g., a ABI 7700 fluorescence reader). The QC assay uses two no target blanks as negative controls and five untyped genomic samples as targets. For consistency, every SNP set is tested with the same genomic samples. In preferred embodiment, the ADS system is responsible for tracking tubes through the QC module. Thus, in some embodiments, if a tube is missing, the ADS program discards, reorders, or searches for the missing tube.

In some preferred embodiments, the user chooses which QC method to run. The operator then chooses how many sets are needed. Then, in some embodiments, the application auto-selects the correct number of SNPs based on priority and prints output (picklist). If a picklist needs to be regenerated, the operator inputs which picklist they are replacing as well as which sets are not valid. The system auto-selects the valid SNPs plus replacement SNPs and print output. Additionally, in some embodiments, picklists are manually generated by SNP number.

The auto-selected SNPs are then removed from being listed as available for auto-selection. In some embodiments, the software prints the following items: SNP/Oligo list (picklist), SNP/Oligo layout (rack setup). The operator then takes the picklist into inventory and removes the completed oligonucleotide sets. In some embodiments, a completed set is unavailable. In this case, the operator regenerates a picklist. Then, in preferred embodiments, the missing SNP set or tube is flagged in the system. Once a picklist is full, the oligonucleotides are moved to the next step.

In some embodiments, the operator then takes the rack setup generated by the picklist and loads the rack. Alternatively, a robotic handling system loads the rack. In preferred embodiments, tubes are scanned as they are placed onto the rack. The scan checks to make sure it is the correct tube and displays the location in the rack where the tube is to be placed.

Completed racks are then placed in a holding area to await the robot prep and robot run. Then, in some embodiments, the operator views what racks are in the queue and determines what genomics and reagent stock will be loaded onto the robot. The robot is then programmed to perform a specific method. Additionally, in some embodiments, the robot or operator records genomics and reagents lot numbers.

In preferred embodiments, a carousel location map is printed that outlines where racks are to be placed. The operator then loads the robot carousel according to the method layout. The rack is scanned (e.g., by the operator or by the ADS program). If the rack is not valid for the current robot method, the operator will be informed. The carousel location for the rack is then displayed. The output plates are then scanned (e.g., by the operator or by the ADS program). If the plate is not valid for the current method the operator is informed. The carousel location for the plate is then displayed.

Then, in some embodiments, the robot is run. The robot then places the plates onto heatblocks for a period of time specified in the method. In some embodiments, the robot then scans the plates on the Cytofluor. Output from the cytofluor is read into the database and attached to the output plate record.

In other embodiments, the output is read on the ABI 7700 real time instrument. In some embodiments, the operator loads the plate on to the 7700. Alternatively, in other embodiments, the robot loads the plate onto the ABI 7700. A scan is then started using the 7700 software. When the scan is completed the output file is saved onto a computer hard drive. The operator then starts the application and scans in the plate bar code. The software instructs the user to browse to the saved output file. The software then reads the file into the database and deletes the file (or tells the operator to delete the file).

The plate reader results (e.g., from a Cytofluor or a ABI 7700) are then analyzed (e.g., by a software program or by the operator). Additionally, in some embodiments, the operator reviews the results of the software analysis of each SNP and takes one of several actions. In some embodiments, the operator approves all automated actions. In other embodiments, the operator reviews and approves individual actions. In some embodiments, the operator marks actions as needing additional review. Alternatively, in other embodiments, the operator passes on reviewing anything. Additionally, in some embodiments, the operator overrides all automated actions.

Depending on the results of the QC analysis, one of several actions is next taken. If the software marks ready for Full Fill, the operator forwards discards diluted Probe/INVADER oligonucleotide mixes and forwards the samples to the packaging module.

If an oligonucleotide set fails quality control, the data is interpreted to determine the cause of the failure. The course of action is determined by such data interpretation. If the software marks an oligonucleotide Reassess Failed Oligonucleotide, no action by user is required, the reassess is handled by automation. In the software marks an oligonucleotide Redilute Failed Oligonucleotide, the operator discards diluted tubes. No other action is required. If the software marks an oligonucleotide Order Target Oligonucleotide, no action by user is required. In this case, a synthetic target oligonucleotide is ordered for further testing. If the software marks an oligonucleotide Fail Oligo(s) Discard Oligo(s), the operator discards the diluted tubes and un-diluted tubes. No other action is required. If the software marks an oligonucleotide Fail SNP, the operator discards the diluted and un-diluted tubes. No other action is required. If the software marks an oligonucleotide Full SNP Redesign, the operator discards the diluted and un-diluted tubes. No other action is required. If the software marks an oligonucleotide Partial SNP Redesign the operator discards diluted tubes and discards some un-diluted tubes. No other action is required.

In some embodiments, the software marks an oligonucleotide Manual Intervention. This step occurs if the operator or software has determined the SNP requires manual attention. This step puts the SNP “on hold” in the tracking system while the operator investigates the source of the failure.

When a set of oligonucleotides (e.g., a INVADER assay set) is completed, the set is transferred to the packaging station.

4. Packaging Component

In some embodiments, one or more components generated using the system of the present invention are packaged using any suitable means. In some embodiments, the packaging system is automated. In some embodiments, the packaging component is controlled by the centralized control network of the present invention.

5. Centralized Control Network

In some embodiments, the automated DNA production process further comprises a centralized control system. In some embodiments, the centralized control system comprises a computer system.

In some embodiments, the computer system comprises computer memory or a computer memory device and a computer processor. In some embodiments, the computer memory (or computer memory device) and computer processor are part of the same computer. In other embodiments, the computer memory device or computer memory are located on one computer and the computer processor is located on a different computer. In some embodiments, the computer memory is connected to the computer processor through the Internet or World Wide Web. In some embodiments, the computer memory is on a computer readable medium (e.g., floppy disk, hard disk, compact disk, DVD, etc). In other embodiments, the computer memory (or computer memory device) and computer processor are connected via a local network or intranet. In certain embodiments, the computer system comprises a computer memory device, a computer processor, an interactive device (e.g., keyboard, mouse, voice recognition system), and a display system (e.g., monitor, speaker system, etc.).

In preferred embodiments, the systems and methods of the present invention comprise a centralized control system, wherein the centralized control system comprises a computer tracking system (tracking software). As discussed above, the items to be manufactured (e.g. oligonucleotide probes, targets, etc) are subjected to a number of processing steps (e.g. synthesis, purification, quality control, etc). Also as discussed above, various components of a single order (e.g. one type of SNP detection kit) are manufactured in separate tubes, and may be subjected to a different number of processing steps. Consequently, the present invention provides systems and methods for tracking the location and status of the items to be manufactured such that multiple components of a single order can be separately manufactured and brought back together at the appropriate time. The tracking system and methods of the present invention also allow for increased quality control and production efficiency.

In some embodiments, the computer tracking system comprises a central processing unit (CPU) and a central database. The central database is the central repository of information about manufacturing orders that are received (e.g. SNP sequence to be detected, final dilution requirements, etc), as well as manufacturing orders that have been processed (e.g. processed by software applications that determine optimal nucleic acid sequences, and applications that assign unique identifiers to orders). Manufacturing orders that have been processed may generate, for example, the number and types of oligonucleotides that need to be manufactured (e.g. probe, INVADER oligonucleotide, synthetic target), and the unique identifier associated with the entire order as well as unique identifiers for each component of an order (e.g. probe, INVADER oligonucleotide, etc). In certain embodiments, the components of an order proceed through the manufacturing process in containers that have been labeled with unique identifiers (e.g. bar coded test tubes, color coded test tubes, etc.).

In certain embodiments, the computer tracking system further comprises one or more scanning units capable of reading the unique identifier associated with each labeled container. In some embodiments, the scanning units are portable (e.g. hand held scanner employed by an operator to scan a labeled container). In other embodiments, the scanning units are stationary (e.g. built into each module). In some embodiments, at least one scanning unit is portable and at least one scanning unit is stationary (e.g. hand held human implemented device).

Stationary scanning units may, for example, collect information from the unique identifier on a labeled container (i.e. the labeled container is ‘red’) as it passes through part of one of the production modules. For example, a rack of 100 labeled containers may pass from the purification module to the dilute and fill module on a conveyor belt or other transport means, and the 100 labeled containers may be read by the stationary scanning unit. Likewise, a portable scanning unit may be employed to collect the information from the labeled containers as they pass from one production module to the next, or at different points within a production module. The scanning units may also be employed, for example, to determine the identity of a labeled container that has been tested (e.g. concentration of sample inside container is tested and the identity of the container is determined).

The scanning units are capable of transmitting the information they collect from the labeled containers to a central database. The scanning units may be linked to a central database via wires, or the information may be transmitted to the central database. The central database collects and processes this information such that the location and status of individual orders and components of orders can be tracked (e.g. information about when the order is likely to complete the manufacturing process may be obtained from the system). The central database also collects information from any type of sample analysis performed within each module (e.g. concentration measurements made during dilute and fill module). This sample analysis is correlated with the unique identifiers on each labeled container such that the status of each labeled container is determined. This allows labeled containers that are unsatisfactory to be removed from the production process (e.g. information from the central database is communicated to robotic or human container handlers to remove the unsatisfactory sample). Likewise, containers that are automatically removed from the production process as unsatisfactory may be identified, and this information communicated to a central database (e.g. to update the status of an order, allow a re-order to be generated, etc). Allowing unsatisfactory samples to be removed prevents unnecessary manufacturing steps, and allows the production of a replacement to begin as early as possible.

As mentioned above, the tracking system of the present invention allows the production of single orders that have multiple components that may proceed through different production modules, and/or that may be processed (at least in part) in separate containers. For example, an order may be for the production of an INVADER assay detection kit. An INVADER assay detection kit is composed of at least 2 components (the INVADER oligonucleotide, and the downstream probe), and generally includes a second downstream probe (e.g. for a different allele), and one or two synthetic targets so controls may be run (i.e. an INVADER assay kit may have 5 separate oligonucleotide sequences that need to be generated). The generation of separate sequences, in separate containers, generally necessitates that the tracking system track the location and status of each container, and direct the proper association of completed oligonucleotides into a single container or kit. Providing each container with a unique identifier corresponding to a single type of oligonucleotide (e.g. an INVADER oligonucleotide), and also corresponding to a single order (a SNP detection kit for diagnosing a certain SNP) allows separate, high through-put manufacture of the various components of a kit without confusion as to what components belong with each kit.

Tracking the location and status of the components of a kit (e.g. a kit composed of 5 different oligonucleotides) has many advantages. For example, near the end of the purification module HPLC is employed, and a simple sample analysis may be employed on each sample in each container to determine if a sample is collected in each tube. If no sample is collected after HPLC is performed, the unique identifier on the container, in connection with the central database, identifies the type of sample that should have been produced (e.g. INVADER oligonucleotide) and a re-order is generated. Identification of this particular oligonucleotide allows the manufacturing process for this oligonucleotide to start over from the beginning (e.g. this order gets priority status over other orders to begin the manufacturing process again). Importantly, the other components of the order may continue the manufacturing process without being discarded as part of a defective order (e.g. the manufacturing process may continue for these oligonucleotides up to the point where the defective oligonucleotide is required). Likewise, additional manufacturing resources are not wasted on the defective component (i.e. additional reagents and time are not spent on this portion of the order in further manufacturing steps).

The unique identifier on each of the containers allows the various components of a given order to be grouped together at a step when this is required (likewise, there is no need to group the components of an order in the manufacturing process until it is required). For example, prior to the dilute and fill module, the various components of a single order may be grouped together such that the contents of the proper containers are combined in the proper fashion in the dilute and fill module. This identification and grouping also allows re-orders to ‘find’ the other components of a particular order. This type of grouping, for example, allows the automated mixing, in the dilute and fill stage, of the first and second downstream probes with the INVADER oligonucleotide, all from the same order. This helps prevent human errors in reading containers and accidentally providing probes intended for one SNP being labeled as specific for a different SNP (i.e. this helps prevent components of different kits from being accidentally mixed together). The identification of individual containers not only allows for the proper grouping of the various components of a single order, but also allows for an order to be customized for a particular customer (e.g. a certain concentration or buffer employed in the second dilute and fill procedure). Finally, containers with finished products in them (e.g. containers with probes, and containers with synthetic targets) need to be associated with each other so they are properly assayed in the quality control module, and packaged together as a single kit (otherwise, quality control and/or a final end-user may find false negative and false positives when attempting to test/use the kit). The ability to track the individual containers allows the components of a kit to be associated together by directing a robot or human operator what tubes belong together. Consequently, final kits are produced with the proper components. Therefore, the tracking systems and methods of the present invention allow high through-put production of kits with many components, while assuring quality production.

6. Production in Practice

This Example describes the production of an INVADER assay kit for SNP detection using the automated DNA production system of the present invention.

A. Oligonucleotide Design

The sequence of the SNP to be detected is first submitted through the automated web-based user interface or through e-mail. The sequences are then transferred to the INVADER CREATOR software. The software designs the upstream INVADER oligonucleotide and downstream probe oligonucleotide. The sequences are returned to the user for inspection. At this point, the sequences are assigned a bar code and entered into the automated tracking system. The bar codes of the probe and INVADER oligonucleotide are linked so that their synthesis, analysis, and packaging can be coordinated.

B. Oligonucleotide Synthesis

Once the probe and INVADER oligonucleotide sequences have been designed, the sequences are transferred to the synthesis component. The bar codes are read and the sequences are logged into the synthesis module. Each module consists of 14 MOSS EXPEDITE 16-channel DNA synthesizers (PE Biosystems, Foster City, Calif.), that prepare the primary probes, and two ABI 3948 48-Channel DNA synthesizers (PE Biosystems, Foster City, Calif.), that prepare the INVADER oligonucleotides. Synthesizing a set of two primary and INVADER probes is complete 3-4 hours. The instruments run 24 h/day. Following synthesis, the automating tracking system reads the bar codes and logs the oligonucleotides as having completed the synthesis module.

The synthesis room is equipped with centralized reagent delivery. Acetonitrile is supplied to the synthesizers through stainless steel tubing. De-blocking solution (3% TCA in methylene chloride) is supplied through Teflon tubing. Tubing is designed to attach to the synthesizers without any modification of the synthesizers. The synthesis room is also equipped with an automated waste removal system. Waste containers are equipped with ventilation and contain sensors that trigger removal of waste through centralized tubing when the cache pots are full. Waste is piped to a centralized storage facility equipped with a blow out wall. The pressure in the synthesis instruments is controlled with argon supplied through a centralized system. The argon delivery system includes local tanks supplied from a centralized storage tank.

During synthesis, the efficiency of each step of the reaction is monitored. If an oligonucleotide fails the synthesis process, it is re-synthesized. The bar coding system scans the container of the oligonucleotide and marks it as being sent back for re-synthesis.

Following synthesis, the oligonucleotides are transported to the cleavage and deprotection station. At this stage, completed oligonucleotides are subjected to a final deprotection step and are cleaved from the solid support used for synthesis. The cleavage and deprotection may be performed manually or through automated robotics. The oligonucleotides are cleaved from the solid support used for synthesis by incubation with concentrated NaOH and collected. The cleavage step takes 12 hours. Following cleavage, the bar code scanner scans the oligonucleotide tubes and logs them as having completed the cleavage and deprotection step.

C. Purification

Following synthesis and cleavage, probe oligonucleotides are further purified using HPLC. INVADER oligonucleotides are not purified, but instead proceed directly to desalting (see below).

HPLC is performed on instruments integrated into banks (modules) of 8. Each HPLC module consists of a Leap Technologies 8-port injector connected to 8 automated Beckman-Coulter HPLC instruments. The automatic Leap injector can handle four 96-well plates of cleaved and deprotected primary probes at a time. The Leap injector automatically loads a sample onto each of the 8 HPLCs.

Buffers for HPLC purification are produced by the automated buffer preparation system. The buffer prep system is in a general access area. Prepared buffer is then piped through the wall in to clean room (HEPA environment). The system includes large vat carboys that receive premeasured reagents and water for centralized buffer preparation. The buffers are piped from central prep to HPLCs. The conductivity of the solution in the circulation loop is monitored as a means of verifying both correct content and adequate mixing. The circulation lines are fitted with venturis for static mixing of the solutions; additional mixing occurs as solutions are circulated through the piping loop. The circulation lines are fitted with 0.05 μm filters for sterilization and removal of any residual particulates.

Each purified probe is collected into a 50-ml conical tube in a carrying case in the fraction collector. Collection is based on a set method, which is triggered by an absorbance rate change within a predetermined time window. The HPLC is run at a flow rate of 5-7.5 ml/min (the maximum rate of the pumps is 10 ml/min.) and each column is automatically washed before the injector loads the next sample. The gradient used is described in Tables 3 and 4 and takes 34 minutes to complete (including wash steps to prepare the column for the next sample). When the fraction collector is full of eluted probes, the tubes are transferred manually to customized racks for concentration in a Genevac centrifugal evaporator. The Genevac racks, containing dry oligonucleotide, are then transferred to the TECAN Nap10 column handler for desalting.

D. Desalting

Following HPLC purification (probe oligonucleotides) or cleavage (INVADER oligonucleotides), oligonucleotides move to the desalting station. The dried oligonucleotides are resuspended in a small volume of water. Desalting steps are performed by a TECAN robot system. The racks used in Genevac centrifugation are also used in the desalting step, eliminating the need for transfer of tubes at this step. The racks are also designed to hold the different sizes of desalting columns, such as the NAP-5 and NAP-10 columns. The TECAN robot loads each oligonucleotide onto an individual NAP-5 or NAP-10 column, supplies the buffer, and collects the eluate.

E. Dilution

Following desalting, the oligonucleotides are transferred to the dilute and fill module for concentration normalization and dispenation. Each module consists of three automated probe dilution and normalization stations. Each station consists of a network-linked computer and a Biomek 2000 interfaced with a SPECTRAMAX spectrophotometer Model 190 or PLUS 384 (Molecular Devices Corp., Sunnyvale Calif.) in a HEPA-filtered environment.

The probe and INVADER oligonucleotides are transferred onto the Biomek 2000 deck and the sequence files are downloaded into the Biomek 2000. The Biomek 2000 automatically transfers a sample of each oligonucleotide to an optical plate, which the spectrophotometer reads to measure the A260 absorbance. Once the A260 has been determined, an Excel program integrated with the Biomek software uses the measured absorbance and the sequence information to calculate the concentration of each oligonucleotide. The software then prepares a dilution table for each oligonucleotide. The probe and INVADER oligonucleotide are each diluted by the Biomek to a concentration appropriate for their intended use. The instrument then combines and dispenses the probe and INVADER oligonucleotides into 1.5 ml microtubes for each SNP set. The completed set of oligonucleotides contains enough material for 5,000 SNP assays.

If an oligonucleotide fails the dilution step, it is first re-diluted. If it again fails dilution, the oligonucleotide is re-purified or returned for re-synthesis. The progress of the oligonucleotide through the dilution module is tracked by the bar coding system. Oligonucleotides that pass the dilution module are scanned as having completed dilution and are moved to the next module.

F. Quality Control

Before shipping, the SNP set is subjected to a quality control assay in a SAGIAN CORE System (Beckman Coulter), which is read on a ABI 7700 real time fluorescence reader (PE Biosystems). The QC assay uses two no target blanks as negative controls and five untyped genomic samples as targets.

The quality control assay is performed in segments. In each segment, the operator or automated system performs the following steps: log on; select location; step specific activity; and log off. The ADS system is responsible for tracking tubes. If a tube is missing, existing ADS program routines will be used to discard/reorder/search for the tube.

In the first step, a picklist is generated. The list includes the identity of the SNPs that are being tested and the QC method chosen. The tubes containing the oligonucleotide are selected by the automated software and a copy of the picklist is printed. The tubes are removed from inventory by the operator and scanned with the bar code reader and being removed from inventory.

The operator or the automated system then takes the rack setup generated by the picklist and loads the rack. Tubes are scanned as they are placed onto the rack. The scan checks to make sure it is the correct tube and displays the location in the rack where the tube is to be placed. Completed racks are placed in a holding area to await the robot prep and robot run.

The operator or the automated system then chooses the genomics and reagent stock to be loaded onto the robot. The robot is programmed with the specific method for the SNP set generated. Lot numbers of the genomics and reagents are recorded. Racks are placed in the proper carousel location. After all the carousel locations have been loaded the robot is run.

Places are then incubated on the robot. The plates are placed onto heatblocks for a period of time specified in the method. The operator then takes the plate and loads it into the ABI 7700. A scan is started using the 7700 software. When the scan is completed the operator transfers the output file onto a Macintosh computer hard drive. The then starts the analysis application and scans in the plate bar code. The software instructs the operator to browse to the saved output file. The software then reads the file into the database and deletes the file.

The results of the QC assay are then analyzed. The operator scans plate in at workstation PC and reviews automated analysis. The automated actions are performed using a spreadsheet system. The automated spreadsheet program returns one of the following results:

1) Mark SNP Oligonucleotide ready for full fill (Operator discards diluted Probe/INVADER mixes. Requires no other action).

2) ReAssess Failed Oligonucleotide (Requires no action by operator, handled by automation).

3) Redilute Failed Oligonucleotide (Operator discards diluted tubes. Requires no other action).

4) Order Target Oligonucleotide (Requires no action by operator, handled by automation).

5) Fail Oligo(s) Discard Oligo(s) (Operator discards diluted tubes. Operator discards un-diluted tubes. Requires no other action).

6) Fail SNP (Operator discards diluted tubes. Operator discards un-diluted tubes. Requires no other action).

7) Full SNP Redesign (Operator discards diluted tubes. Operator discards un-diluted tubes. Requires no other action).

8) Partial SNP Redesign (Operator discards diluted tubes. Operator discards some un-diluted tubes. Requires no other action).

9) Manual Intervention (This step occurs if the operator or software has determined the SNP requires manual attention. This step puts the SNP “on hold” in the tracking system).

The operator then views each SNP analysis and either approves all automated actions, approves individual actions, marks actions as needing additional review, passes on reviewing anything, or over rides automated actions. Once the SNP set has passed the QC analysis, the oligonucleotides are transferred to the packaging station.

All publications and patents mentioned in the above specification are herein incorporated by reference. Various modifications and variations of the described method and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention which are obvious to those skilled in the relevant fields are intended to be within the scope of the following claims.

We claim:

1. A method for selecting an optimal coupling temperature of a nucleic acid synthesis reaction comprising:
a) providing:
i. a nucleic acid synthesizer comprising a heating component; and
ii. nucleic acid synthesis reagents;
b) synthesizing a plurality of nucleic acid molecules with said synthesizer and said nucleic acid synthesis reagents, wherein the synthesis of each nucleic acid molecule of said plurality of nucleic acid molecules comprises a coupling reaction performed at a different temperature within a temperature range; and
c) measuring nucleic acid synthesis efficiency for each of said syntheses of said plurality of nucleic acid molecules; and
d) selecting an optimal coupling temperature within said temperature range.

2. The method of claim 1, wherein said temperature range is 20 to 60 degrees C.

3. A nucleic acid synthesizer comprising one or more reaction chambers and a heating component configured to heat said one or more reaction chambers during a synthesis reaction.

4. The nucleic acid synthesizer of claim 3, wherein said heating component comprises a resistance heater.

5. The nucleic acid synthesizer of claim 3, wherein said heating component comprises a Peltier device.

6. The nucleic acid synthesizer of claim 3, wherein said heating component comprises a heated reagent.

7. The nucleic acid synthesizer of claim 3, wherein said heating component comprises a magnetic induction device.

8. The nucleic acid synthesizer of claim 3, wherein said heating component comprises microwaves.

9. The nucleic acid synthesizer of claim 3, wherein said heating component comprises a transfer of heat from a fluid or a gas.

10. A nucleic acid synthesizer, comprising:
a. one or more reaction chambers containing an oligonucleotide; and
b. a heating component,
wherein said heating component is configured to heat said one or more reaction chambers during a synthesis reaction wherein said oligonucleotide is coupled to a synthesis reagent.
